(19) United States
(12) Patent Application Publication
(10) Pub. No.: US 2024/0207290 A1
(43) Pub. Date: Jun. 27, 2024

(54) CYCLIC PHOSPHONATE COMPOSITION AND PREPARATION METHOD THEREOF

(71) Applicant: GANNEX PHARMA CO., LTD., Shanghai (CN)

(72) Inventors: Xuyu Chai, Shanghai (CN); Jinzi Jason Wu, Shanghai (CN)

(21) Appl. No.: 18/555,993

(22) PCT Filed: Mar. 18, 2022
(86) PCT No.: PCT/US22/71221

§ 371 Date: Oct. 18, 2023

(30) Foreign Application Priority Data
Feb. 24, 2022 (CN) 202210172544.0

(51) Int. Cl. A61K 31/665; A61K 9/107; A61K 9/20; A61K 9/48

(52) U.S. Cl. CPC A61K 31/665; A61K 9/1075; A61K 9/2009; A61K 9/2013; A61K 9/2018; A61K 9/2031; A61K 9/2054; A61K 9/2095; A61K 9/4808; A61K 9/4816

(57) ABSTRACT

A pharmaceutical composition comprising: a compound of formula (1)

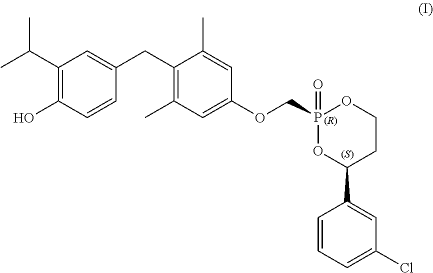

and a polymer material, wherein the compound of formula (1) maintains a supersaturated concentration in the pharmaceutical composition and is suitable for storage at room temperature.

### CROSS REFERENCE TO RELATED APPLICATIONS

This application is a national stage of International Application No. PCT/US22/71221, filed Mar. 18, 2022, which claims priority to Chinese Application CN 202210172544.0, filed Feb. 24, 2022. The entirety of the aforementioned applications is incorporated herein by reference.

### FIELD

The present application generally relates to the field of pharmaceutical preparations, and particularly relates to a suitable cyclic phosphonate pharmaceutical composition and a preparation method thereof.

### BACKGROUND

A cyclic phosphonate compound shown in formula (I) (molecular formula: C28H32ClO5P, molecular weight: 514.98, CAS number: 852948-13-1) is a novel oral thyroid hormone β receptor agonist (THR-beta agonist) which is expected to be used for fatty liver diseases5.

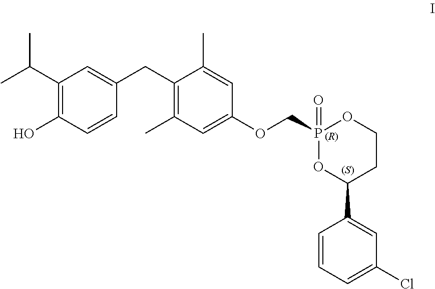

Chinese patent application 202010105909.9 discloses a solubilizing composition of a compound shown in formula (I) and a preparation method thereof. Semi-solid capsules prepared by the technology need to be stored at a temperature lower than room temperature (no more than 15° C.), which adds inconvenience to storage and carrying. Chinese patent application 202010227177.0 discloses another composition of the compound shown in (I) and its preparation method. The pharmaceutical preparation prepared by this technology can be stored stably at room temperature (no more than 30° C.), but there is also the problem of short maintenance time of supersaturated concentration. It can be seen from the results in Table 7 of Example 2 of the patent that the dissolution of each formulation in Examples G1-L1 at 360 min is only 38.9%˜76.3% of the highest point, and some drugs are precipitated quickly from supersaturated aqueous solutions.

Dissolution of drugs is a prerequisite for oral absorption into the body to exert curative effect. After oral administration, drugs usually stay in the mouth and esophagus for less than 1 min, in the stomach for 0.5˜2 h, in the small intestine for 6˜8 h, in the large intestine for 10˜20 h, and in the whole digestive tract for as long as 24 h or more. Therefore, maintaining stable supersaturated concentration for a longer time by preparation technology is helpful to achieve solubilization and synergism.

### SUMMARY

One aspect of the present application relates to a pharmaceutical composition that comprises by weight:
(1) 1 part of a compound of formula (I)

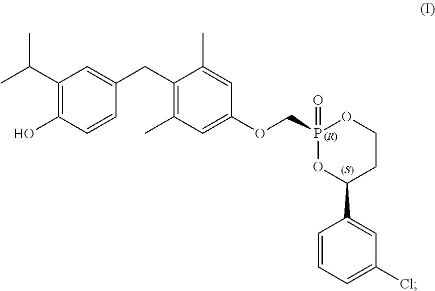

(2) 9 to 45 parts of polymer material,
wherein the compound of formula (I) maintains a supersaturated concentration in the pharmaceutical composition and is suitable for storage at room temperature.

In some embodiments, the pharmaceutical composition is in the form of an amorphous solid dispersion prepared by hot melt extrusion, wherein the pharmaceutical composition spontaneously forms nano micelles, wherein the compound of formula (I) is stably distributed in the hydrophobic interior of the nanomicelles or the hydrophobic interior of the membrane of the nanomicelles due to its water insolubility and high lipid solubility, so as to maintain a stable supersaturated concentration in the pharmaceutical composition.

In some embodiments, the compound of formula (I) is formulated in a stabilizing formulation that allows the compound to be stored at room temperature a stable supersaturated concentration for at least 3, 4, 5 or 6 months.

In some embodiments, the pharmaceutical composition comprises by weight 1 part of the compound of formula (I) and 9-15, 9-20, 9-25, 9-30, 9-35, 9-40, 15-20, 15-25, 15-30, 15-35, 15-40, 15-45, 20-25, 20-30, 20-35, 20-40, 20-45, 25-30, 25-35, 25-40, 25-45, 30-35, 30-40, 35-45, 35-40, 35-45 or 40-45 parts of polymer material.

In some embodiments, the polymer material comprises one or more of the polymers selected from the group consisting of polyvinyl lactam polymers, cellulose derivative polymers, polyacrylic resins, polyethylene glycol, polyoxyethylene and polyvinyl alcohol.

In some embodiments, the polymer material comprises one or more polyvinyl lactam polymers selected from the group consisting of polyvinylpyrrolidone (PVP), polyvinylpyrrolidone (PVP/VA) and polyethylene caprolactam-polyvinyl acetate-polyethylene glycol graft copolymer (Soluplus)®. In some embodiments, the polymer material comprises polyethylene caprolactam-polyvinyl acetate-polyethylene glycol graft copolymer. In some embodiments, the polymer material consists of polyethylene caprolactam-polyvinyl acetate-polyethylene glycol graft copolymer.

In some embodiments, the polymer material comprises one or more cellulose derivative polymers selected from the group consisting of methyl cellulose, ethyl cellulose, hydroxypropyl methylcellulose (HPMC), hydroxypropyl cellulose (HPC), methyl hydroxyethyl cellulose (MHEC), hydroxypropyl methylcellulose succinate (HPMC-AS), hydroxypropyl methylcellulose phthalate (HPMCP) and cellulose acetate phthalate (CAP).

In some embodiments, the polymer material comprises one or more polyacrylic resins selected from the group consisting of methacrylic acid copolymers, methacrylic acid-ethyl acrylate copolymers, amino methacrylic acid copolymers and methacrylate copolymers.

In some embodiments, the pharmaceutical composition further comprises one or more non-polymeric material. In some embodiments, the pharmaceutical composition further comprises one or more non-polymeric material selected from the group consisting of pharmaceutically acceptable pharmaceutical excipients, nonvolatile weak acids, neutral or weakly acidic inorganic substances, and pharmaceutically acceptable excipients with a melting point lower than 80° C.

In some embodiments, the pharmaceutical composition further comprises one or more pharmaceutically acceptable pharmaceutical excipients with a melting point lower than 80° C., selected from the group consisting of lipid materials, such as triethyl citrate, acetylated triethyl citrate, propylene glycol biskwai ester, medium chain triglyceride, monoglyceride, monooleate, diethylene glycol monoethyl ether, hydrogenated castor oil, myristyl alcohol, hexadecanol, hexadecanol, stearyl alcohol, palmitic acid, palmitoyl alcohol, palmitoyl alcohol, palmitoyl alcohol, 2,6-di-tert-butyl-p-cresol, vitamin E; poloxamer 188, poloxamer 407, polyethylene glycol 15-hydroxystearate (Kolliphor® HS15), sodium dodecyl sulfate, propylene glycol monooctanoate, polyethylene glycol oleate (Labrafac™ lipophile WL1349), propylene glycol monolaurate, lauric acid polyglycerol ester (Gelucire® 44/14), stearic acid polyglycerol ester (Gelucire® 50/13), caprylic acid polyglycerol ester (Labrasol®), sorbitan fatty acid ester polyoxyethylene ether (Tween), sorbitan fatty acid ester (Span) and vitamin E polyglycol Succinate®.

In some embodiments, the pharmaceutical composition does not contain other non-polymeric materials.

In some embodiments, the compound of formula (I) is prepared by spray drying, solvent evaporation or hot melt extrusion. In some embodiments, the compound of formula (I) is prepared by spray drying or hot melt extrusion. In some embodiments, the compound of formula (I) is prepared by hot melt extrusion.

In some embodiments, the compound of formula (I) is present in the pharmaceutical composition of the present application in a crystalline form, in an amorphous form, or in a part-crystalline/part amorphous form.

In some embodiments, the amorphous form of the compound of formula (I) is present in the pharmaceutical composition of the present application in an amount of 50% or greater by weight of the total amount of the compound of formula (I).

In some embodiments, the amorphous form of the compound of formula (I) is present in the pharmaceutical composition of the present application in an amount of 80% or greater by weight of the total amount of the compound of formula (I).

In some embodiments, the amorphous form of the compound of formula (I) is present in the pharmaceutical composition of the present application in an amount of 90% or greater by weight of the total amount of the compound of formula (I).

In some embodiments, the pharmaceutical composition of the present application further comprises one or more pharmaceutically acceptable excipients such as diluents, adhesives, disintegrants and lubricants.

In some embodiments, the pharmaceutical composition of the present application is in the form of particles or powders. In some embodiments, the pharmaceutical composition of the present application is processed into capsules or tablets, or formulated into particles or granules and packaged into bags, or processed into suspensions.

Another aspect of the present application relates to a process or method for preparing the pharmaceutical composition of the present application. In some embodiments, the process or method comprises the step of extruding a mixture comprising by weight (1) 1 part of a compound of formula (I) and (2) 9 to 45 parts of polymer material by hot melt extrusion to form an extruded product, wherein the compound of formula (I) maintains a supersaturated concentration in the extruded product at room temperature.

In some embodiments, the compound of formula (I) maintains a supersaturated concentration in the extruded product for at least 3, 4, 5, or 6 months at room temperature.

In some embodiments, the extruded product is crushed or cut into particles or powders. In some embodiments, the particles or powders are packaged into capsules or bags, or are pressed into tablets. In some embodiments, the particles or powders are packaged into hydroxypropyl methylcellulose capsules.

In certain embodiments, the pharmaceutical composition of the present application is formulated in a fixed dose tablet or capsule that contains 1-15 mg of the compound of formula (I).

In certain embodiments, the fixed dose tablet or capsule contains 2.5 mg or 5 mg of the compound of formula (I).

An aspect of the application is a method for treatment of disease in a patient in need thereof, comprising: administering to the patient an effective amount of the pharmaceutical composition described herein.

In certain embodiments, the disease is selected from the group consisting of nonalcoholic fatty liver disease (NAFLD) and non-alcoholic steatohepatitis (NASH).

### BRIEF DESCRIPTION OF THE DRAWINGS

FIG. 1 shows a dissolution curve (n=6) of prescription samples of compositions A1 to C1 in Example 1,

FIG. 2 shows a dissolution curve (n=6) of a sample formulated according to composition a2 in Example 1

While the present disclosure will now be described in detail, and it is done so in connection with the illustrative embodiments, it is not limited by the particular embodiments illustrated in the figures and the appended claims.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

### I. Definitions

“Administering” means providing a pharmaceutical agent or composition to a subject, and includes, but is not limited to, administering by a medical professional and self-administering.

Administration of the compounds disclosed herein or the pharmaceutically acceptable salts thereof, or the additional therapeutic agents disclosed herein can be via any of the accepted modes of administration for agents that serve similar utilities including, but not limited to, orally, subcutaneously, intravenously, intranasally, topically, transdermally, intraperitoneally, intramuscularly, intrapulmonarily, vaginally, rectally, or intraocularly. Oral and parenteral administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“in combination” or “combination” refers to the compound of formula (I) and at least one additional therapeutic agent being substantially effective in the body at a same time. Both can be be administered substantially at the same time, or both can be administered at different times but have effect on the body at the same time. For example, “in combination” includes administering the compound of formula (I) before the administration of the at least one additional therapeutic agent, and subsequently administering the at least one additional therapeutic agent while functioning of the compound of formula (I) in the body is substantially extant. In addition, “in combination” includes administering the at least one additional therapeutic agent before the administration of the compound of formula (I), and subsequently administering the compound of formula (I) while functioning of the at least one additional therapeutic agent in the body is substantially extant. When a pharmaceutical composition is described as containing the compound of formula (I) and the at least one additional therapeutic agent in combination, this term refers to both agents being concurrently present in the composition. The terms “in combination” and “combination” may further relate to the advantageous use of the compound of formula (I) and the at least one additional therapeutic agent in the absence of concomitant treatment for liver diseases such as NAFLD or NASH.

“Active pharmaceutical ingredient” means the substance in a pharmaceutical composition that provides a desired therapeutic effect.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable excipient” or “pharmaceutically acceptable carrier” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “unit dosage form” refers to a composition containing an amount of a compound that is suitable for administration to a subject, in a single dose, according to good medical practice. However, as further described below, the preparation of a single or unit dosage form however, does not imply that the dosage form is administered once per day or once per course of therapy.

A “loading dose” refers to an initial dose of a compound which is higher than subsequent doses.

A “maintenance dose” refers to a subsequent dose that follows a loading dose, and occurs later in time than a loading dose. One of ordinary skill in the art will be aware that the dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. As used herein, the period of administration of the maintenance dose may be referred to as the “maintenance phase” of the treatment period.

The phrase, “mode of administration” refers to the means by which a compound is administered to a subject. As such, the phrase encompasses the dosage form (for example, a tablet, powder, dissolved liquid, suspension, emulsion, aerosol, etc.) and the mechanism by which the dosage form is applied to the subject (for example, by oral administration or injection). The “mode of administration” may further encompass the dose, dose amount, and dosing schedule by which a compound is administered to a subject. The phrase “duration of the treatment” refers to the time commencing with administration of the first dose and concluding with the administration of the final dose, such length of time being determined by one of ordinary skill in the art of treating a given disease.

The phrase “dosing holiday” refers to a period of 24 hours or more during which either no dose is administered to the subject, or a reduced dose is administered to the subject.

“Fatty liver diseases” and liver disorders include the primary fatty liver diseases, steatosis or nonalcoholic fatty liver (NAFL), non-alcoholic fatty liver disease (NAFLD), non-alcoholic steatohepatitis (NASH), and hepatocellular carcinoma (HCC). Fatty liver diseases are typically conditions wherein large vacuoles of triglyceride fat accumulate in liver cells via the process of steatosis (i.e., abnormal retention of lipids within a cell). Accumulation of fat may also be accompanied by a progressive inflammation of the liver (hepatitis), called steatohepatitis. By considering the contribution of alcohol, fatty liver disease may be termed alcoholic steatosis or non-alcoholic fatty liver disease (NAFLD).

“Nonalcoholic fatty liver disease (NAFLD)” is an umbrella term for a range of liver conditions affecting people who drink little to no alcohol. As the name implies, the main characteristic of NAFLD is too much fat stored in liver cells. NAFLD is increasingly common around the world, especially in Western nations. In the United States, it is the most common form of chronic liver disease, affecting about one-quarter of the population. Some individuals with NAFLD can develop “nonalcoholic steatohepatitis (NASH),” an aggressive form of fatty liver disease, which is marked by liver inflammation and may progress to advanced scarring (cirrhosis) and liver failure. This damage is similar to the damage caused by heavy alcohol use.

“Subject” as used herein, means a human or a non-human mammal, including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

A “subject suspected of having” means a subject exhibiting one or more clinical indicators of a disease or condition.

A “subject in need thereof” means a subject identified as in need of a therapy or treatment.

### II. Pharmaceutical Composition

One aspect of the present application relates to a pharmaceutical composition that comprises by weight 1 part of a compound of formula (I)

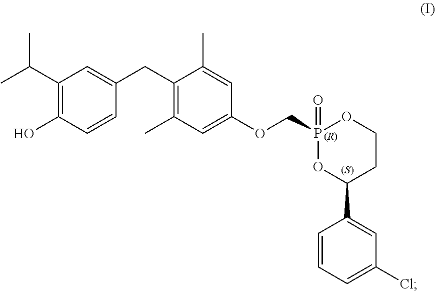

and 3 to 90 parts of polymer material, wherein the compound of formula (I) is present at a supersaturated concentration in the pharmaceutical composition. The pharmaceutical composition is stable at room temperature for an extended period of time.

In some embodiments, the pharmaceutical composition is in the form of an amorphous solid dispersion prepared by hot melt extrusion, wherein the pharmaceutical composition spontaneously forms nano micelles, wherein the compound of formula (I) is distributed in the hydrophobic portion of the nanomicelles due to its water insolubility and high lipid solubility, so as to maintain a stable supersaturated concentration in the pharmaceutical composition.

In some embodiments, the compound of formula (I) is formulated in a stabilizing formulation that allows the compound to be stored at room temperature a stable supersaturated concentration for at least 3, 4, 5 or 6 months.

In some embodiments, the pharmaceutical composition comprises by weight 1 part of the compound of formula (I) and 9-15, 9-20, 9-25, 9-30, 9-35, 9-40, 9-45, 15-20, 15-25, 15-30, 15-35, 15-40, 15-45, 20-25, 20-30, 20-35, 20-40, 20-45, 25-30, 25-35, 25-40, 25-45, 30-35, 30-40, 35-45, 35-40, 35-45 or 40-45 parts of polymer material.

In some embodiments, the polymer material comprises one or more of the polymers selected from the group consisting of polyvinyl lactam polymers, cellulose derivative polymers, polyacrylic resins, polyethylene glycol, polyoxyethylene and polyvinyl alcohol.

In some embodiments, the polymer material comprises one or more polyvinyl lactam polymers selected from the group consisting of polyvinylpyrrolidone (PVP), polyvinylpyrrolidone (PVP/VA) and polyethylene caprolactam-polyvinyl acetate-polyethylene glycol graft copolymer (Soluplus)®. In some embodiments, the polymer material comprises polyethylene caprolactam-polyvinyl acetate-polyethylene glycol graft copolymer. In some embodiments, the polymer material consists of polyethylene caprolactam-polyvinyl acetate-polyethylene glycol graft copolymer.

In some embodiments, the polymer material comprises one or more cellulose derivative polymers selected from the group consisting of methyl cellulose, ethyl cellulose, hydroxypropyl methylcellulose (HPMC), hydroxypropyl cellulose (HPC), methyl hydroxyethyl cellulose (MHEC), hydroxypropyl methylcellulose succinate (HPMC-AS), hydroxypropyl methylcellulose phthalate (HPMCP) and cellulose acetate phthalate (CAP).

In some embodiments, the polymer material comprises one or more polyacrylic resins selected from the group consisting of methacrylic acid copolymers, methacrylic acid-ethyl acrylate copolymers, amino methacrylic acid copolymers and methacrylate copolymers.

In some embodiments, the pharmaceutical composition further comprises one or more non-polymeric material. In some embodiments, the pharmaceutical composition further comprises one or more non-polymeric material selected from the group consisting of pharmaceutically acceptable pharmaceutical excipients, nonvolatile weak acids, neutral or weakly acidic inorganic substances, and pharmaceutically acceptable excipients with a melting point lower than 80° C.

In some embodiments, the pharmaceutical composition further comprises one or more pharmaceutically acceptable pharmaceutical excipients with a melting point lower than 80° C., selected from the group consisting of lipid materials, such as triethyl citrate, acetylated triethyl citrate, propylene glycol biskwai ester, medium chain triglyceride, monoglyceride, monooleate, diethylene glycol monoethyl ether, hydrogenated castor oil, myristyl alcohol, hexadecanol, hexadecanol, stearyl alcohol, palmitic acid, palmitoyl alcohol, palmitoyl alcohol, palmitoyl alcohol, 2,6-di-tert-butyl-p-cresol, vitamin E; poloxamer 188, poloxamer 407, polyethylene glycol 15-hydroxystearate (Kolliphor® HS15), sodium dodecyl sulfate, propylene glycol monooctanoate, polyethylene glycol oleate (Labrafac™ lipophile WL 1349), propylene glycol monolaurate, lauric acid polyglycerol ester (Gelucire® 44/14), stearic acid polyglycerol ester (Gelucire® 50/13), caprylic acid polyglycerol ester (Labrasol®) sorbitan fatty acid ester polyoxyethylene ether (Tween), sorbitan fatty acid ester (Span) and vitamin E polyglycol Succinate®.

In some embodiments, the pharmaceutical composition comprises by weight 1 part of the compound of formula (I) and 17-27, 19-25, 21-23 parts of polymer material. In some embodiments, the polymer material comprises polyethylene caprolactam-polyvinyl acetate-polyethylene glycol graft copolymer.

In some embodiments, the pharmaceutical composition comprises by weight 1 part of the compound of formula (I) and 28-38, 30-36, 32-34 parts of polymer material. In some embodiments, the polymer material comprises polyethylene caprolactam-polyvinyl acetate-polyethylene glycol graft copolymer. In some embodiments, the pharmaceutical composition further comprises 0.2-5 parts of triethyl citrate. In some embodiments, the pharmaceutical composition further comprises 1 part of triethyl citrate.

In some embodiments, the pharmaceutical composition does not contain other non-polymeric materials.

In some embodiments, the compound of formula (I) is prepared by spray drying, solvent evaporation or hot melt extrusion. In some embodiments, the compound of formula (I) is prepared by spray drying or hot melt extrusion. In some embodiments, the compound of formula (I) is prepared by hot melt extrusion.

In some embodiments, the compound of formula (I) is present in the pharmaceutical composition of the present application in a crystalline form, in an amorphous form, or in a part-crystalline/part amorphous form.

In some embodiments, the amorphous form of the compound of formula (I) is present in the pharmaceutical composition of the present application in an amount of 50% or greater by weight of the total amount of the compound of formula (I).

In some embodiments, the amorphous form of the compound of formula (I) is present in the pharmaceutical composition of the present application in an amount of 80% or greater by weight of the total amount of the compound of formula (I).

In some embodiments, the amorphous form of the compound of formula (I) is present in the pharmaceutical composition of the present application in an amount of 90% or greater by weight of the total amount of the compound of formula (I).

In some embodiments, the pharmaceutical composition of the present application further comprises one or more pharmaceutically acceptable excipients such as diluents, adhesives, disintegrants and lubricants.

Exemplary substances, which can serve as pharmaceutically-acceptable excipients, include sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; tale; solid lubricants, such as stearic acid and magnesium stearate; calcium sulfate; vegetable oils, such as peanut oil, cottonseed oil, sesame oil, olive oil, corn oil, and theobroma oil; polyols, such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often than once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Oral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethylcellulose, AVICEL RC-591, tragacanth and sodium alginate; typical wetting agents include lecithin and polysorbate 80; and typical preservatives include methyl paraben and sodium benzoate. Oral liquid compositions may also contain one or more components, such as sweeteners, flavoring agents and colorants disclosed above.

Such compositions may also be coated by conventional methods, typically with pH or time-dependent coatings, such that the active agent(s) are released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

In some embodiments, the pharmaceutical composition of the present application may optionally include one or more other active agents.

In some embodiments, the pharmaceutical composition of the present application is in the form of particles or powders. In some embodiments, the pharmaceutical composition of the present application is processed into capsules or tablets, or formulated into particles or granules and packaged into bags, or processed into suspensions.

### II. Method of Preparation

Another aspect of the present application relates to a process or method for preparing the pharmaceutical composition of the present application. In some embodiments, the process or method comprises the step of spray drying, solvent evaporation or hot melt extrusing a pre-extrusion mixture comprising by weight (1) 1 part of a compound of formula (1) and (2) 3 to 90 parts of polymer material by hot melt extrusion to form an extruded product, wherein the compound of formula (I) maintains a supersaturated concentration in the extruded product at room temperature.

In some embodiments, the process or method comprises the step of extruding a pre-extrusion mixture comprising by weight (1) 1 part of a compound of formula (I) and (2) 3 to 90 parts of polymer material by hot melt extrusion to form an extruded product, wherein the compound of formula (I) maintains a supersaturated concentration in the extruded product at room temperature. In some embodiments, the extruding step is performed at an extrusion temperature of 80-140° C. or 90-130° C.

In some embodiments, the pre-extrusion mixture comprises by weight 1 part of the compound of formula (I) and 9-15, 9-20, 9-25, 9-30, 9-35, 9-40, 9-45, 15-20, 15-25, 15-30, 15-35, 15-40, 15-45, 20-25, 20-30, 20-35, 20-40, 20-45, 25-30, 25-35, 2540, 2545, 30-35, 30-40, 35-45, 3540, 35-45 or 40-45 parts of polymer material.

In some embodiments, the polymer material comprises one or more of the polymers selected from the group consisting of polyvinyl lactam polymers, cellulose derivative polymers, polyacrylic resins, polyethylene glycol, polyoxyethylene and polyvinyl alcohol.

In some embodiments, the polymer material comprises one or more polyvinyl lactam polymers selected from the group consisting of polyvinylpyrrolidone (PVP), polyvinylpyrrolidone (PVP/VA) and polyethylene caprolactam-polyvinyl acetate-polyethylene glycol graft copolymer (Soluplus)®. In some embodiments, the polymer material comprises polyethylene caprolactam-polyvinyl acetate-polyethylene glycol graft copolymer. In some embodiments, the polymer material consists of polyethylene caprolactam-polyvinyl acetate-polyethylene glycol graft copolymer.

In some embodiments, the polymer material comprises one or more cellulose derivative polymers selected from the group consisting of methyl cellulose, ethyl cellulose, hydroxypropyl methylcellulose (HPMC), hydroxypropyl cellulose (HPC), methyl hydroxyethyl cellulose (MHEC), hydroxypropyl methylcellulose succinate (HPMC-AS), hydroxypropyl methylcellulose phthalate (HPMCP) and cellulose acetate phthalate (CAP).

In some embodiments, the polymer material comprises one or more polyacrylic resins selected from the group consisting of methacrylic acid copolymers, methacrylic acid-ethyl acrylate copolymers, amino methacrylic acid copolymers and methacrylate copolymers.

In some embodiments, the pre-extrusion mixture further comprises one or more non-polymeric material. In some embodiments, the pre-extrusion mixture further comprises one or more non-polymeric material selected from the group consisting of pharmaceutically acceptable pharmaceutical excipients, nonvolatile weak acids, neutral or weakly acidic inorganic substances, and pharmaceutically acceptable excipients with a melting point lower than 80° C.

In some embodiments, the pre-extrusion mixture further comprises one or more pharmaceutically acceptable pharmaceutical excipients with a melting point lower than 80° C., selected from the group consisting of lipid materials, such as triethyl citrate, acetylated triethyl citrate, propylene glycol biskwai ester, medium chain triglyceride, monoglyceride, monooleate, diethylene glycol monoethyl ether, hydrogenated castor oil, myristyl alcohol, hexadecanol, hexadecanol, stearyl alcohol, palmitic acid, palmitoyl alcohol, palmitoyl alcohol, palmitoyl alcohol, 2,6-di-tert-butyl-p-cresol, vitamin E; poloxamer 188, poloxamer 407, polyethylene glycol 15-hydroxystearate (Kolliphor® HS15), sodium dodecyl sulfate, propylene glycol monooctanoate, polyethylene glycol oleate (Labrafac™ lipophile WL1349), propylene glycol monolaurate, lauric acid polyglycerol ester (Gelucire® 44/14), stearic acid polyglycerol ester (Gelucire® 50/13), caprylic acid polyglycerol ester (Labrasol®), sorbitan fatty acid ester polyoxyethylene ether (Tween), sorbitan fatty acid ester (Span) and vitamin E polyglycol succinate.

In some embodiments, the pre-extrusion mixture comprises by weight 1 part of the compound of formula (I) and 17-27, 19-25, 21-23 parts of polymer material. In some embodiments, the polymer material comprises polyethylene caprolactam-polyvinyl acetate-polyethylene glycol graft copolymer.

In some embodiments, the pre-extrusion mixture comprises by weight 1 part of the compound of formula (I) and 28-38, 30-36, 32-34 parts of polymer material. In some embodiments, the polymer material comprises polyethylene caprolactam-polyvinyl acetate-polyethylene glycol graft copolymer. In some embodiments, the pre-extrusion mixture further comprises 0.2-5 parts of triethyl citrate. In some embodiments, the pharmaceutical composition further comprises 1 part of triethyl citrate.

In some embodiments, the pre-extrusion mixture does not contain other non-polymeric materials.

In some embodiments, the compound of formula (I) maintains a supersaturated concentration in the extruded product for at least 3, 4, 5, or 6 months at room temperature.

In some embodiments, the extruded product is crushed or cut into particles or powders. In some embodiments, the particles or powders are packaged into capsules or bags, or are pressed into tablets. In some embodiments, the particles or powders are packaged into hydroxypropyl methylcellulose capsules.

The advantage of the methods and formulations disclosed herein is that the prepared pharmaceutical composition can prolong the maintenance time of supersaturated concentration of the compound shown in formula (I) by forming nano micelles, and thus providing more stable dissolution of the active ingredient in the pharmaceutical composition.

### IV. Method of Treatment

Another aspect of the present application provides methods for reducing the levels of low-density lipoprotein and triglyceride, reducing lipotoxicity, improving liver function and reducing liver fat using the pharmaceutical composition of the present application. The method comprises the step of administering to a subject in need of such treatment an effective amount of the pharmaceutical composition of the present application.

Another aspect of the present application relates to a method for treating or ameliorating s symptom of nonalcoholic steatohepatitis (NASH). The method comprises the step of administering to a subject in need of such treatment an effective amount of the pharmaceutical composition of the present application.

In some embodiments, the subject has a fatty liver disease or a fibrotic disorder and/or inflammatory condition affecting the liver.

In some embodiments, the subject has a secondary fatty liver disease, such as alcoholic liver disease (ALD), fatty liver associated with chronic hepatitis infection, total parental nutrition (TPN), Reye's Syndrome, as well as gastrointestinal disorders, such as intestinal bacterial overgrowth (IBO), gastroparesis, irritable bowel (IBS) disorders, and the like.

In certain preferred embodiments, the disease for treatment with the compositions described herein is liver fibrosis, such as steatosis, non-alcoholic fatty liver disease (NAFLD), non-alcoholic steatohepatitis (NASH), alcoholic steatohepatitis (ASH), and hepatocellular carcinoma (HCC).

In some embodiments, the subject for treatment has NAFLD. In some embodiments, the subject has diabetes. In some embodiments, the subject has type 2 diabetes. In some embodiments, the subject has type 1 diabetes. In certain embodiments, the subject with NAFLD has type 2 diabetes mellitus (T2DM). In other embodiments, the subject with NAFLD has metabolic syndrome (MS).

In some embodiments, the subject has a metabolic disease or disorder. Exemplary metabolic diseases or disorders for treatment with the compositions of the present application include diabetes, metabolic syndrome, obesity, hyperlipidemia, high cholesterol, arteriosclerosis, hypertension, NASH, NAFL, NAFLD, hepatic steatosis, and any combination thereof.

In some embodiments, the subject has metabolic syndrome (MS). In some embodiments, the subject has one or more of these diseases or disorders. In some embodiments, the subject is at risk of developing one or more of these diseases.

In some embodiments, the subject has insulin resistance, increased blood glucose concentrations, high blood pressure, elevated cholesterol levels, elevated triglyceride levels, or is obese.

In some embodiments, the subject has polycystic ovary syndrome.

In some embodiments, the patient being treated is at risk of developing liver fibrosis or cirrhosis.

In some embodiments, the fibrosis comprises non-cirrhotic hepatic fibrosis.

In some embodiments, the liver fibrosis is advanced.

In some embodiments, the disease effects tissue selected from the group consisting of liver, kidney, skin, epidermis, endodermis, muscle, tendon, cartilage, heart, pancreas, lung, uterus, nervous system, testis, penis, ovary, adrenal gland, artery, vein, colon, intestine (e.g. small intestine), biliary tract, soft tissue (e.g. mediastinum or retroperitoneum), bone marrow, joint and stomach fibrosis, in particular liver, gut, lung, heart, kidney, muscle, skin, soft tissue, bone marrow, intestinal, eye and joint fibrosis.

In some embodiments, the disease is selected from the group consisting of metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn's disease, ulcerative colitis, keloid, old myocardial infarction, scleroderma/systemic sclerosis, inflammatory diseases, neurodegenerative diseases, cancers, liver cancer, hepatocallular carcinoma, gastrointestinal cancer, gastric cancer, meningioma associated with neurofibromatosis, pancreatic neuroendocrine tumors, pancreatic exocrine tumors, leukemia, myeloproliferative/myelodisplastic diseases, mastocytosis, dermatofibrosarcoma, solid tumors including breast, lung, thyroid or colorectal cancer, a prostate cancer, liver fibrosis or cirrhosis of any origin, metabolic disease-induced liver fibrosis or cirrhosis, NAFLD-induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn's disease-induced fibrosis, ulcerative colitis-induced fibrosis, intestine (e.g. small intestine) fibrosis, colon fibrosis, stomach fibrosis, skin fibrosis, epidermis fibrosis, endodermis fibrosis, skin fibrosis due to scleroderma/systemic sclerosis, lung fibrosis, lung fibrosis consecutive to chronic inflammatory airway diseases, such as COPD, asthma, emphysema, smoker's lung, tuberculosis, pulmonary fibrosis, idiopathic pulmonary fibrosis (IPF), heart fibrosis, kidney fibrosis, nephrogenic systemic fibrosis, muscle fibrosis, soft tissue (e.g. mediastinum or retroperitoneum) fibrosis, bone marrow fibrosis, joint fibrosis, tendon fibrosis, cartilage fibrosis, pancreas fibrosis, uterus fibrosis, nervous system fibrosis, testis fibrosis, ovary fibrosis, adrenal gland fibrosis, artery fibrosis, vein fibrosis, eye fibrosis, endomyocardial fibrosis, mediastinal fibrosis, myelofibrosis, retroperitoneal fibrosis, progressive massive fibrosis (a complication of coal workers' pneumoconiosis), proliferative fibrosis, neoplastic fibrosis, peri-implantational fibrosis and asbestosis, arthrofibrosis, adhesive capsulitis.

In some embodiments, the disease is selected from the group consisting of metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn's disease, ulcerative colitis, liver cancer, hepatocallular carcinoma, gastrointestinal cancer, gastric cancer, colorectal cancer, metabolic disease-induced liver fibrosis or cirrhosis, NAFLD-induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn's disease-induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker's lung, tuberculosis, pulmonary fibrosis, idiopathic pulmonary fibrosis (IPF).

The methods described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in e.g., Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

In some embodiment, the method further comprises the step of administering to the subject an effective amount of an additional therapeutic agent.

In various embodiments, the additional therapeutic agent is selected from the group consisting of one or more of a farnesoid X receptor (FXR) agonist, a peroxisome proliferator-activated receptor (PPAR) agonist, an anti-diabetic agent, an antifibrosis compound, an anti-oxidant, an anti-inflammatory compound, a lipid lowering agent, a fish oil derivative, an analogue thereof, a pegylated variant thereof, or any combination of the foregoing.

In other embodiments, the additional therapeutic agent is selected from the group consisting of one or more of a fibrate, a bile acid receptor modulator, an anti-inflammatory compound, an antifibrotic compound, a GLP-1 (Glucagon like peptide-1) agonist, a metabolic modulator, fish oil derivatives, diacylglycerol acyltransferase (DGAT) inhibitors, or any combination of the foregoing.

In other embodiments, the additional therapeutic agent is an agent that increases insulin secretion. In another example, the additional therapeutic agent is an agent that increases the sensitivity of target cells, tissues, or organs to insulin. In another example, the at least one additional therapeutic agent is an agent that decreases the level of glucose in the blood.

In other embodiments, the at least one additional therapeutic agent is insulin or an insulin analog. In a further example, the insulin or insulin analog is selected from Humulin® R, insulin lispro (Humalog®), insulin aspart (Novolog®), insulin glulisine (Apidra®), Prompt insulin zinc (Semilente®), insulin glargine (Lantus®), insulin detemir (Levemir®), Isophane insulin, insulin zinc (Lente®), extended insulin zinc (Ultralente®), insulin degludec, Exubera®, and Afrezza®.

In other embodiments, the additional therapeutic agent is an inhibitor of the ATP-sensitive K+ channel in the pancreatic beta cells. In a further example, the at least one additional therapeutic agent is a sulfonylurea. In a further example, the sulfonylurea is selected from tolbutamide (Orinase®), acetohexamide (Dymelor), tolazamide (Tolinase®), chlorpropamide (Diabinese®), carbutamide (Glucidoral®), metahexamide, glipizide (Glucotrol®), glyburide or glibenclamide (Micronase®), glycopyramide, gliquidone (Glurenorm), gliclazide (Uni Diamicron), glibornuride, glisoxepide, glimepiride (Amaryl®), and JB253 (Broichhagen et al., Nature Comm. 5, Article No. 5116 (2014)). In another further example, the at least one additional therapeutic agent is selected from meglitinide, repaglinide (Prandin®), nateglinide (Starlix®), mitiglinide, and linogliride.

In other embodiments, the additional therapeutic agent is an agonist of FFA1/GPR40 (Free Fatty acid Receptor 1). In a further example, the FFA1/GPR40 agonist is fasiglifam.

In other embodiments, the additional therapeutic agent is an incretin mimetic. In a further example, the incretin mimetic is a glucagon-like peptide-1 (GLP-1) or agonist of the GLP-1 receptor thereof. In a further example, the GLP-1 receptor agonist is selected from exenatide/exendin-4, liraglutide, taspoglutide, lixisenatide, albiglutide, dulaglutide, semaglutide, BRX-0585 (Pfizer/Biorexis), and CJC-1 134-PC (exendin-4 conjugated to human albumin). In another further example, the incretin mimetic is a gastric inhibitory peptide (GIP) or GIP analog.

In other embodiments, the additional therapeutic agent is an inhibitor of dipeptidyl peptidase-4 (DPP-4, also known in the art as DPP-IV). In a further example, the DPP-4 inhibitor is selected from vildagliptin (Galvus®), sitagliptin (Januvia®), saxagliptin (Onglyza®), linagliptin (Tradjenta®), alogliptin, septagliptin, anagliptin, gemigliptin, teneligliptin, carmegliptin, gosogliptin, dutogliptin, berberine, and lupeol.

In other embodiments, the additional therapeutic agent is a human peroxisome proliferator activated receptor (PPAR) gamma agonist. In a further example, the PPAR gamma agonist is selected from thiazolidinediones and glitazones, e.g., rosiglitazone, troglitazone, pioglitazone, englitazone, balaglitazone, rivoglitazone, ciglitazone, lobeglitazone, and netoglitazone.

In other embodiments, the additional therapeutic agent is a biguanide. In a further example, the biguanide is selected from metformin, buformin, and phenformin.

In other embodiments, the additional therapeutic agent is a bile acid sequestrant. In a further example, the bile acid sequestrant is selected from anion exchange resin, quaternary amines (e.g., cholestyramine or colestipol), and an ileal bile acid transporter inhibitor.

In other embodiments, the additional therapeutic agent is an agent that facilitates metabolism of glucose (e.g., phosphorylation of glucose). In one example, the at least one additional therapeutic agent is a glucokinase activator. In a further example, the glucokinase activator is a compound as described in WO 2000/058293.

In other embodiments, the additional therapeutic agent is an agent that blocks renal reabsorption of glucose. In one example, the at least one additional therapeutic agent is a SGLT-2 inhibitor. In a further example, the SGLT-2 inhibitor is selected from canagliflozin, dapagliflozin, empagliflozin, remogliflozin, sergliflozin, tofogliflozin, ipragliflozin, and ertugliflozin.

In other embodiments, the additional therapeutic agent is an agent that reduces glucose absorption in the intestine. In one example, the at least one additional therapeutic agent is an alpha-glucosidase inhibitor. In a further example, the alpha-glucosidase inhibitor is selected from miglitol (Glyset®), acarbose (Precose®), and voglibose

In other embodiments, the additional therapeutic agent is an agent that slows gastric emptying and/or suppresses glucagon. In one example, the at least one additional therapeutic agent is an amylin or amylin analog. In a further example, the amylin analog is pramlintide.

In other embodiments, the additional therapeutic agent is a microsomal triglyceride transfer protein (MTP) inhibitor. In a further example, the MTP inhibitor is selected from midaglizole, isaglidole, deriglidole, idazoxan, efaroxan, and fluparoxan.

In other embodiments, the additional therapeutic agent is selected from bromocriptine, benfluorex, and tolrestat.

In other embodiments, the additional therapeutic agent are bile acid FXR agonists, including, obeticholic acid (OCA) and INT-767. Non-bile acid FXR agonists include cilovexor (GS-9674), tropifexor (LJN-452), nidufexor (LMB763), and EDP-305. Additional FXR agonists include analogues, pegylated variants, and combinations of the foregoing FXR agonists.

In other embodiments, the additional therapeutic agent are PPAR agonists, including, but are not limited to GW 9578, GW 7647, GW 590735, and GFT505. PPAR agonists include PPAR-alpha (PPAR-α) agonists, PPAR-gamma (PPAR-γ) agonists, PPAR-epsilon (PPAR-S) agonists, dual PPAR-α/γ agonists, dual PPAR-α/δ agonists, and pan-PPAR agonists targeting all three PPAR isozyme (i.e., α/β/γ). Exemplary PPAR-α agonists also include bezafibrate, fenofibrate, pemafibrate, gemfibrozil, clofibrate, and omega-3 poly-unsaturated fatty acids (Ω-PUFAs), such as Omacor. PPAR-γ agonists include, but are not limited to pioglitazone, lobeglitazone, rosiglitazone, INTI31, MSDC-0602K, and GW501516. An exemplary PPAR-5 agonist is seladelpar, GW501516; an exemplary dual PPARα/γ agonists is saroglitazar; an exemplary dual PPAR-α/δ agonist is elafibranor; exemplary pan-PPAR agonists include lanifibranor, netoglitazone, GW677964, DRL-605, and GW25019. Additional PPAR agonists include analogues, pegylated variants, and combinations of the foregoing PPAR agonists.

In other embodiments, the additional therapeutic agent are fibrates, including, but are not limited to fenofibrate, fenofibric acid, gemfibrozil, clofibrate, pemafibrate, clofibrate, gemfibrozil, ciprofibrate, bezafibrate, ABT-335, etofibrate, pirifibrate, beclofibrate, analogs thereof, pegylated variants thereof, and combinations thereof.

In other embodiments, the additional therapeutic agent are GLP-1 (Glucagon like peptide-1) agonists, including, but are not limited to dulaglutide, exenatide, liraglutide, albiglutide, lixisenatide, semaglutide, insulin glargine, engineered analogs thereof, pegylated variants thereof, and combinations thereof.

In other embodiments, the additional therapeutic agent are metabolic modulators, including, but are not limited to thyroid hormone receptor agonists, selective androgen receptor modulators, mitochondrial membrane transport protein modulators, selective estrogen receptor modulators, stearoyl-CoA desaturase 1 (SCD1) inhibitors, dipeptidyl peptidase 4 (DPP-4) inhibitors, inhibitors of sodium glucose cotransporters 1 and/or 2 (SGLT1, SGLT2, or dual SGLT1/SGLT2 inhibitors), recombinant fibroblast growth factor 19 (FGF19), recombinant fibroblast growth factor 21 (FGF21), engineered analogs thereof, pegylated variants thereof, and combinations thereof.

In other embodiments, the additional therapeutic agent are fish oil derivatives, including, but are not limited to omega-3-fatty acid alkyl esters, including omega-3-fatty acid ethyl esters, such as ethyl (5Z,8Z,11Z,14Z, 17Z)-eicosa-5, 8,11,14, 17-pentaenoate, ethyl (4Z,7 Z, 10Z, 13Z, 16Z, 19Z)-docosa-4,7, 10, 13, 16, 19-hexaenoate, ethyl (72, 10Z, 13Z, 16Z, 19Z)-docosapentaenoate, ethyl hexadecatrienoate, α-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, and nisinic acid ethyl ester. In other embodiments, the fish oil derivative is an omega-3-fatty acid trigylyceride.

In other embodiments, the additional therapeutic agent are diacylglycerol acyltransferase (DGAT) inhibitors, including, but are not limited to the compounds disclosed in U.S. Pat. No. 8,962,618.

In other embodiments, the additional therapeutic agent are anti-diabetic agents, including, but are not limited to incretin hormone agonists, including glucagon-like peptide 1 receptor agonists (GLP-1RAs), GLP-IRAs include dulaglutide, semaglutide, exenatide, liraglutide, albiglutide, lixisenatide, semaglutide, insulin glargine, glucagon (GCG) and its agonists, and glucose-dependent insulinotropic polypeptide (GIP) agonists; dipeptidyl peptidase 4 (DPP4) inhibitors, DPP4 inhibitors include sitagliptin and vildagliptin; inhibitors of sodium glucose cotransporters 1 and/or 2 (SGLT1, SGLT2, and dual SGLT1/SGLT2 inhibitors), SGLT2 inhibitors include dapagliflozin, empagliflozin, canagliflozin, ipragliflozin, luseogliflozin, licogliflozin (LIK066; dual SGLT1/2); oral insulin, as well as dual or triple agonists thereof. An exemplary GLP-1/GCG receptor dual agonist is cotadutide (MEDI0382). Exemplary GLP-1/GIP receptor dual agonists include CT868 and trizepatide (LY3298176). An exemplary GLP-1/GCG/GIP triple agonist is HM15211. An exemplary dual GLP-1/FGF21 agonist is YH25724. Additional anti-diabetic drugs include metformin, pioglitazone, and rosiglitazone, as well as analogues, pegylated variants, and combinations of the foregoing anti-diabetic agents.

In some embodiments, the additional therapeutic agent is selected from the group consisting of one or more of an anti-fibrotic drug selected from CCR2 and/or CCR5 antagonists, such as cenicriviroc (dual CCR2/CCR5 antagonist); apoptosis signal-regulating kinase 1 (ASK1) inhibitors, such as selonsertib; angiotensin receptor blockers (ARBs), such as losartan; transforming growth factor-β (TGF-β) inhibitors, such as galunisertib; fibroblast growth factor 19 (FGF19) and FGF19 analogs, such as NGM282; FGF21 and FGF21 analogs, such as pegbelfermin (BMS-986036), PF-05231023, AKR-001 and B1089-100; agonistic anti-FGFR1c/KLB antibodies, such as NGM313 (MK-3655) and BFKB8488A; Takeda G protein-coupled receptor 5 (TGR5) activators, such as INT-777; RDX8940; galectin-3 antagonists, such as belapectin (GR-MD-02) and GB1211; Hsp47 antagonists, such as ND-L02-s0201 siRNA; anti-lysyl oxidase-like 2 (LOXL-2) mAbs, such as simtuzumab; IL-11 inhibitors, as well as analogues, pegylated variants, and combinations thereof.

In other embodiments, the additional therapeutic agent is selected from the group consisting of one or more of an an anti-fibrotic drug selected from receptor tyrosine kinase inhibitors (RTKIs), such as nintedanib and sorafenib; angiotensin II (ATI) receptor blockers, a connective tissue growth factor (CTGF) inhibitor, or antifibrotic compound susceptible to interfere with the TGF.beta.- and BMP-activated pathways including activators of the latent TGF.beta. complex such as MMP2, MMP9, THBS1 or cell-surface integrins, TGF-β receptors type I (TGFBRI) or type II (TGFBRII) and their ligands, such as TGF-β, Activin, inhibin, Nodal, anti-Mullerian hormone, GDFs and BMPs; auxiliary co-receptors (also known as type III receptors); components of the SMAD-dependent canonical pathway, including regulatory or inhibitory SMAD proteins; members of the SMAD-independent or non-canonical pathways, including various branches of MAPK signaling, TAK1, Rho-like GTPase signaling pathways, phosphatidylinositol-3 kinase/AKT pathways, and TGF-β-induced epithelial-mesenchymal transition (EMT) processes; canonical and non-canonical Hedgehog signaling pathways, including Hh ligands; canonical and non-canonical wingless-type (wnt) and Notch signaling pathway inhibitors members, including those subject to TGF-β signaling; pirfenidone; nintedanib; a collagenase, such as Clostridium histolyticum collagenase; steroids (e.g., corticosteroids, such as prednisone); BMP9 and/or BMP10 antagonists; immune-suppressing and/or anti-inflammatory agents, such as gamma-interferon, cyclophosphamide, azathioprine, methotrexate, penicillamine, cyclosporine, colchicine, antithymocyte globulin, mycophenolate mofetil, and hydroxychloroquine; calcium channel blockers (e.g., nifedipine); para-aminobenzoic acid (PABA); dimethyl sulfoxide; pan caspase inhibitors; TGF-β signaling modifiers, such as relaxin, SMAD7, HGF, and BMP7, as well as TGF-β1, TGF-β RI, TGF-βR II, EGR-1, and CTGF inhibitors; cytokine and cytokine receptor antagonists (inhibitors of IL-1β, IL-5 IL-6, IL-13, IL-21, IL-4R, IL-13Rα1, GM-CSF, TNFα, oncostatin M, WISP-1, and PDGFs), cytokines and chemokines, such as IFN-γ, IFN-α/β, IL-12, IL-10, HGF, CXCL10, and CXCL11; chemokine antagonists, including inhibitors of CXCL1, CXCL2, CXCL12, CCL2, CCL3, CCL6, CCL17, and CCL18; chemokine receptor antagonists, including inhibitors of CCR2, CCR3, CCR5, CCR7, CXCR2, and CXCR4; TLR antagonists, including inhibitors of TLR3, TLR4, and TLR9; angiogenesis antagonists, such as VEGF-specific antibodies and adenosine deaminase replacement therapy, antihypertensive drugs, including beta blockers and inhibitors of ANG II, angiotensin converting enzyme (ACE), and aldosterone; vasoactive substances, such as ET-1 receptor antagonists and bosetan; inhibitors of enzymes that synthesize and process collagen, including inhibitors of prolyl hydroxylase; B cell antagonists, such as rituximab; integrin/adhesion molecule antagonists that block α1β1 and αvβ6 integrins, as well as inhibitors of integrin linked kinase; antibodies and small molecule inhibitors against ICAM-1 or VCAM-1; proapoptotic drugs targeting myofibroblasts; MMP inhibitors of MMP2, MMP9, or MMP12; antibodies and small molecule inhibitors against TIMP-1.

In other embodiments, the additional therapeutic agent are antioxidants, including, but are not limited to vitamin E, glutathione (GSH), L-glutamyl-L-cysteinyl-glycine, ursodeoxycholic acid (UDCA), resveratrol, silymarin, metadoxine, as well as analogues, pegylated variants, and combinations thereof.

In other embodiments, the additional therapeutic agent are anti-inflammatory compounds, including, but are not limited to phosphodiesterase (PDE) inhibitors and/or tumor necrosis factor-alpha (TNF-α) inhibitors, such as pentoxifylline (PTX); L-carnitine; seloncertib; tipelukast; vitamin D3; G protein-coupled receptor 84 (GRP84); ursodeoxycholic acid (UDCA); vascular adhesion protein-1 (VAP-1)/semicarbazide-sensitive amine oxidase (SSAO) inhibitors, such as BI 1467335 (PXS-4728A), UP-1586, and LJP-1207; caspase inhibitors, such as emricasan and GS-9450; toll-like receptor (TLR)-4 antagonists, such as JKB-121; nucleotide-binding and oligomerization domain (NOD)-like receptor (NLR) inhibitors, such as the NLR family pyrin domain containing 3 (NLRP3) inhibitor, MCC950; JAK/STAT inhibitors, glucocorticoids, NSAIDS, cyclophosphamide, nitrosoureas, folic acid analogs, purine analogs, pyrimidine analogs, methotrexate, azathioprine, mercaptopurine, ciclosporin, myriocin, tacrolimus, sirolimus, mycophenolic acid derivatives, fingolimod and other sphingosine-1-phosphate receptor modulators, monoclonal and/or polyclonal antibodies against such targets as proinflammatory cytokines and proinflammatory cytokine receptors, T-cell receptor, and integrins, analogues therefrom; pegylated variants thereof; and combinations thereof.

In other embodiments, the additional therapeutic agent are lipid lowering agents, including, but are not limited to ezetimibe; HMG-CoA reductase inhibitors (statins), including lipophilic statins, such as atorvastatin, simvastatin, lovastatin and fluvastatin, and hydrophilic statins, such as rosuvastatin, pravastatin and pitavastatin; stearoyl-CoA desaturase 1 (SCD-1) inhibitors, such as the compound of formula I; acetyl-CoA carboxylase (ACC) inhibitors, such as GS-0976, PF-05221304, PF-05175157, NDI-010976, firsocostat, ND-630 and ND-654; diacylglycerol O-acyltransferase-2 (DGAT-2) inhibitors, such as PF-06865571 and IONIS-DGAT2rx; fatty acid synthase (FAS) inhibitors, such as TVB-2640 and FT-4101.

In other embodiments, the additional therapeutic agent are fish oil derivatives, including, but are not limited to omega-3-fatty acid alkyl esters, including omega-3-fatty acid ethyl esters, such as ethyl (5Z,8Z,11Z,14Z, 17Z)-eicosa-5, 8,11,14, 17-pentaenoate, ethyl (4Z,7 Z, 10Z, 13Z, 16Z, 19Z)-docosa-4,7, 10, 13, 16, 19-hexaenoate, ethyl (72, 10Z, 13Z, 16Z, 19Z)-docosapentaenoate, ethyl hexadecatrienoate, a-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, and nisinic acid ethyl ester. In other embodiments, the fish oil derivative is an omega-3-fatty acid trigylyceride.

In other embodiments, the additional therapeutic agent is Aramchol (3-arachidylamido-7a,12a-dihydroxy-5-cholan-24-oic acid), which is a bile acid/fatty acid conjugate comprising an arachidic acid moiety and a cholic acid moiety. Aramchol is described in U.S. Pat. Nos. 6,384,024, 6,395,722, 6,589,946, 7,501,403, 8,110,564 and 8,975,246, incorporated herein by reference.

In other embodiments, the additional therapeutic agent is a VDR agonist selected from the group consisting of calciferol, alfacalcidol, 1,25-dihydroxyvitamin D3, Vitamin D2, Vitamin D3, calcitriol, Vitamin D4, Vitamin D5, dihydrotachysterol, calcipotriol, tacalcitol 1,24-dihydroxyvitamin D3 and paricalcitol.

In other embodiments, the additional therapeutic agent is one or more of Acetyl-CoA carboxylase inhibitors; Adenosine A3 receptor agonists; Aldosterone antagonists and Mineralocorticoid antagonists; AMP activated protein kinase stimulator; Amylin receptor agonist and Calcitonin receptor agonists; Angiopoietin-related protein-3 inhibitors; Anti-LPS antibodies; Apical sodium-codependent bile acid transporter inhibitors; Betaine anhydrous or RM-003; bioactive lipids; Cannabinoid CBI receptor antagonists; Dual cannabinoid CBI receptor/iNOS inhibitor; Caspase inhibitors; Cathepsin inhibitors; CCR antagonists; CCR3 chemokine modulators and eotaxin 2 ligand inhibitors; Diacylglycerol-O-acyltransferase (DGAT) inhibitors; Dipeptidyl peptidase IV (DPP4) inhibitors; Insulin ligand and insulin receptor agonists; Insulin sensitizer and MCH receptor-1 antagonist; NOX (NADPH oxidase) inhibitors, such as dual NOX 1 and 4 inhibitors; Extracellular matrix protein modulators; Stearoyl CoA desaturase-1 inhibitors/fatty acid bile acid conjugates (FABAC); Fatty Acid Synthase (FAS) Inhibitors; Fibroblast Growth Factor 19 (FGF-19) receptor ligands, such as Recombinant Fibroblast Growth Factor 19 (FGF-19) protein, or functional engineered variant of the FGF-19 protein; Fibroblast Growth Factor 21 (FGF-21) receptor ligands such as Fibroblast Growth Factor 21 (FGF-21) protein, or functional engineered variant of the FGF-21 protein; Farnesoid X receptor (FXR) agonists; Galectin 3 inhibitors; Glucagon-like peptide-1 (GLP-1) analogs and GLP-1 receptor agonists; G-protein coupled receptor (GPCR) modulators; G-protein coupled receptor 84 antagonist, connective tissue growth factor ligand inhibitor and Free fatty acid receptor 1 agonists; Hedgehog cell-signaling pathway inhibitors; Integrin inhibitors; ketohexokinase inhibitorsLeukotriene (LTyPhosphodiesterase (PDEyLipoxygenase (LO) inhibitors; Lysyl oxidase homolog 2 inhibitors (LOXL2 inhibitors); Macrolides; Methyl CpG binding protein 2 modulator and Transglutaminase inhibitors; miRNA antagonists; Mitochondrial carrier family inhibitor and Mitochondrial phosphate carrier protein inhibitor; Monoclonal antibodies; Myeloperoxidase inhibitors; mTOR modulators; NAD-dependent deacetylase sirtuin stimulator; PDE S inhibitor; Nicotinic Acid Receptor (GPR109) Agonists; nuclear receptor ligands; P2Y13 protein agonists; Phenylalanine hydroxylase stimulators; Protease-activated receptor (PAR)-2 antagonists; Protein kinase modulators; Rho-associated protein kinase 2(ROCK2) inhibitors; Sodium-GLucose Transport (SGLT) 1 inhibitors; Sodium-glucose transport (SGLT) 2 inhibitors; Stearoyl-CoA desaturase-1 inhibitors; signal-regulating kinase 1 (ASK) inhibitors; thyroid receptor β (THR β) agonists; Toll Like Receptor 2 (TLR-2) antagonists; Toll Like Receptor 4 (TLR-4) antagonists; Type I natural killer T cells inhibitors; Tyrosine kinase receptor (RTK) modulators; Urate anion exchanger 1 inhibitors and Xanthine oxidase inhibitors; Vascular adhesion protein-1 (VAP-1) inhibitors; Acetyl-CoA carboxylase inhibitors; Anti-LPS antibodies; Apical sodium-codependent bile acid transporter inhibitors; bioactive lipids; Cannabinoid CBI receptor antagonists; Dual cannabinoid CBI receptor/iNOS inhibitor; Caspase inhibitors; Cathepsin inhibitors; CCR antagonists; Diacylglycerol-O-acyltransferase (DGAT) inhibitors; Dipeptidyl peptidase IV (DPP4) inhibitors; NOX (NADPH oxidase) inhibitors, such as dual NOX 1 and 4 inhibitors; Extracellular matrix protein modulators; Stearoyl CoA desaturase-1 inhibitors/fatty acid bile acid conjugates (FABAC); Galectin 3 inhibitors; Glucagon-like peptide-1 (GLP-1) analogs; G-protein coupled receptor (GPCR) modulators; Integrin inhibitors; Leukotriene (LT)/Phosphodiesterase (PDE)/Lipoxygenase (LO) inhibitors; Macrolides; miRNA antagonists; Monoclonal antibodies; mTOR modulators; nuclear receptor ligands; P2Y13 protein agonists; Fibroblast Growth Factor 19 (FGF-19) receptor ligands, such as Recombinant Fibroblast Growth Factor 19 (FGF-19) protein, or functional engineered variant of the FGF-19 protein;- Fibroblast Growth Factor 21 (FGF-21) receptor ligands such as Fibroblast Growth Factor 21 (FGF-21) protein, or functional engineered variant of the FGF-21 protein.

Other active agents for use in combination with the compound of formula I of the present application include antibiotics, such as rifaximin, norflocacin and augmentin; mitochondrial-derived peptides, such as MOTS-c and CB4211; growth differentiation factor (GDF15) agonists, such as NGM395, NN-9215 and (LA-GDF15); mineralcorticoid receptor antagonists, such as spironolactone, eplerenone, and apararenone (MT-3995); adipokines, such as leptin, adipoleptin, metreleptin, and osmotin; ileal bile acid transporter (IBAT)/apical sodium-dependent bile acid transporter (ASBT) inhibitors, such as A4250 and volixibat; thyroid hormone receptor-β (THRβ) agonists, such as resmetirom (MGL-3196); TNF-α, inhibitors, such as infliximab and thalidomide; IL-1 receptor antagonists, such as anakinra; probiotics, such as VSL #3 and Lactobacillus rhamnosus GG; mitochondrial membrane transport protein modulators; androgen receptor modulators; estrogen receptor modulators; bicyclol; docosahexanoic acid (DHA); cysteamine bitartrate (CB); PXL065 (DRX-065); orlistat; IL-22; G-CSF; Imm-124E; Pirfenidone, nintedanib, and/or a fibroblast growth factor receptor antagonist, and/or a collagenase, such as Clostridium histolyticum collagenase analogues therefrom; pegylated variants thereof; and combinations thereof.

### Regimen of Administration

When administered in combination with the additional therapeutic agent, the pharmaceutical composition of the present application and the additional therapeutic agents may be administered simultaneously or sequentially.

In some embodiments, dosages are administered every other day for the duration of the treatment. In other embodiments, dosages are administered on two out of every three days for the duration of the treatment. In still other embodiments, dosages are administered two out of every four days for the duration of the treatment.

In some embodiments, dosages are administered daily for one day, followed by a two day dosing holiday. In some embodiments, dosages are administered daily for one day, followed by a two day dosing holiday. In some embodiments, dosages are administered daily for one day, followed by a three day dosing holiday. In some embodiments, dosages are administered daily for one day, followed by a four day dosing holiday. In some embodiments, dosages are administered daily for one day, followed by a five day dosing holiday. In some embodiments, dosages are administered daily for one day, followed by a six day dosing holiday. In some embodiments, dosages are administered daily for one day, followed by a seven day dosing holiday. In some embodiments, dosages are administered daily for one day, followed by an eight day dosing holiday. In some embodiments, dosages are administered daily for one day, followed by a nine day dosing holiday. In some embodiments, dosages are administered daily for one day, followed by a ten day dosing holiday. In some embodiments, dosages are administered daily for one day, followed by an eleven day dosing holiday. In some embodiments, dosages are administered daily for one day, followed by a twelve day dosing holiday. In some embodiments, dosages are administered daily for one day, followed by a thirteen day dosing holiday. In some embodiments, dosages are administered daily for one day, followed by a fourteen day dosing holiday.

In some embodiments, dosages are administered daily for two days, followed by a one day dosing holiday. In some embodiments, dosages are administered daily for two days, followed by a two day dosing holiday. In some embodiments, dosages are administered daily for two days, followed by a three day dosing holiday. In some embodiments, dosages are administered daily for two days, followed by a four day dosing holiday. In some embodiments, dosages are administered daily for two days, followed by a five day dosing holiday. In some embodiments, dosages are administered daily for two days, followed by a six day dosing holiday. In some embodiments, dosages are administered daily for two days, followed by a seven day dosing holiday. In some embodiments, dosages are administered daily for two days, followed by an eight day dosing holiday. In some embodiments, dosages are administered daily for two days, followed by a nine day dosing holiday. In some embodiments, dosages are administered daily for two days, followed by a ten day dosing holiday. In some embodiments, dosages are administered daily for two days, followed by an eleven day dosing holiday. In some embodiments, dosages are administered daily for two days, followed by a twelve day dosing holiday. In some embodiments, dosages are administered daily for two days, followed by a thirteen day dosing holiday. In some embodiments, dosages are administered daily for two days, followed by a fourteen day dosing holiday.

In some embodiments, dosages are administered daily for three days, followed by a one day dosing holiday. In some embodiments, dosages are administered daily for three days, followed by a two day dosing holiday. In some embodiments, dosages are administered daily for three days, followed by a three day dosing holiday. In some embodiments, dosages are administered daily for three days, followed by a four day dosing holiday. In some embodiments, dosages are administered daily for three days, followed by a five day dosing holiday. In some embodiments, dosages are administered daily for three days, followed by a six day dosing holiday. In some embodiments, dosages are administered daily for three days, followed by a seven day dosing holiday. In some embodiments, dosages are administered daily for three days, followed by an eight day dosing holiday. In some embodiments, dosages are administered daily for three days, followed by a nine day dosing holiday. In some embodiments, dosages are administered daily for three days, followed by a ten day dosing holiday. In some embodiments, dosages are administered daily for three days, followed by an eleven day dosing holiday. In some embodiments, dosages are administered daily for three days, followed by a twelve day dosing holiday. In some embodiments, dosages are administered daily for three days, followed by a thirteen day dosing holiday. In some embodiments, dosages are administered daily for three days, followed by a fourteen day dosing holiday.

In some embodiments, dosages are administered daily for four days, followed by a one day dosing holiday. In some embodiments, dosages are administered daily for four days, followed by a two day dosing holiday. In some embodiments, dosages are administered daily for four days, followed by a three day dosing holiday. In some embodiments, dosages are administered daily for four days, followed by a four day dosing holiday. In some embodiments, dosages are administered daily for four days, followed by a five day dosing holiday. In some embodiments, dosages are administered daily for four days, followed by a six day dosing holiday. In some embodiments, dosages are administered daily for four days, followed by a seven day dosing holiday. In some embodiments, dosages are administered daily for four days, followed by an eight day dosing holiday. In some embodiments, dosages are administered daily for four days, followed by a nine day dosing holiday. In some embodiments, dosages are administered daily for four days, followed by a ten day dosing holiday. In some embodiments, dosages are administered daily for four days, followed by an eleven day dosing holiday. In some embodiments, dosages are administered daily for four days, followed by a twelve day dosing holiday. In some embodiments, dosages are administered daily for four days, followed by a thirteen day dosing holiday. In some embodiments, dosages are administered daily for four days, followed by a fourteen day dosing holiday.

In some embodiments, dosages are administered daily for five days, followed by a one day dosing holiday. In some embodiments, dosages are administered daily for five days, followed by a two day dosing holiday. In some embodiments, dosages are administered daily for five days, followed by a three day dosing holiday. In some embodiments, dosages are administered daily for five days, followed by a four day dosing holiday. In some embodiments, dosages are administered daily for five days, followed by a five day dosing holiday. In some embodiments, dosages are administered daily for five days, followed by a six day dosing holiday. In some embodiments, dosages are administered daily for five days, followed by a seven day dosing holiday. In some embodiments, dosages are administered daily for five days, followed by an eight day dosing holiday. In some embodiments, dosages are administered daily for five days, followed by a nine day dosing holiday. In some embodiments, dosages are administered daily for five days, followed by a ten day dosing holiday. In some embodiments, dosages are administered daily for five days, followed by an eleven day dosing holiday. In some embodiments, dosages are administered daily for five days, followed by a twelve day dosing holiday. In some embodiments, dosages are administered daily for five days, followed by a thirteen day dosing holiday. In some embodiments, dosages are administered daily for five days, followed by a fourteen day dosing holiday.

In some embodiments, dosages are administered daily for six days, followed by a one day dosing holiday. In some embodiments, dosages are administered daily for six days, followed by a two day dosing holiday. In some embodiments, dosages are administered daily for six days, followed by a three day dosing holiday. In some embodiments, dosages are administered daily for six days, followed by a four day dosing holiday. In some embodiments, dosages are administered daily for six days, followed by a five day dosing holiday. In some embodiments, dosages are administered daily for six days, followed by a six day dosing holiday. In some embodiments, dosages are administered daily for six days, followed by a seven day dosing holiday. In some embodiments, dosages are administered daily for six days, followed by an eight day dosing holiday. In some embodiments, dosages are administered daily for six days, followed by a nine day dosing holiday. In some embodiments, dosages are administered daily for six days, followed by a ten day dosing holiday. In some embodiments, dosages are administered daily for six days, followed by an eleven day dosing holiday. In some embodiments, dosages are administered daily for six days, followed by a twelve day dosing holiday. In some embodiments, dosages are administered daily for six days, followed by a thirteen day dosing holiday. In some embodiments, dosages are administered daily for six days, followed by a fourteen day dosing holiday.

In some embodiments, dosages are administered daily for seven days, followed by a one day dosing holiday. In some embodiments, dosages are administered daily for seven days, followed by a two day dosing holiday. In some embodiments, dosages are administered daily for seven days, followed by a three day dosing holiday. In some embodiments, dosages are administered daily for seven days, followed by a four day dosing holiday. In some embodiments, dosages are administered daily for seven days, followed by a five day dosing holiday. In some embodiments, dosages are administered daily for seven days, followed by a six day dosing holiday. In some embodiments, dosages are administered daily for seven days, followed by a seven day dosing holiday. In some embodiments, dosages are administered daily for seven days, followed by an eight day dosing holiday. In some embodiments, dosages are administered daily for seven days, followed by a nine day dosing holiday. In some embodiments, dosages are administered daily for seven days, followed by a ten day dosing holiday. In some embodiments, dosages are administered daily for seven days, followed by an eleven day dosing holiday. In some embodiments, dosages are administered daily for seven days, followed by a twelve day dosing holiday. In some embodiments, dosages are administered daily for seven days, followed by a thirteen day dosing holiday. In some embodiments, dosages are administered daily for seven days, followed by a fourteen day dosing holiday.

In some embodiments, dosages are administered daily for eight days, followed by a one day dosing holiday. In some embodiments, dosages are administered daily for eight days, followed by a two day dosing holiday. In some embodiments, dosages are administered daily for eight days, followed by a three day dosing holiday. In some embodiments, dosages are administered daily for eight days, followed by a four day dosing holiday. In some embodiments, dosages are administered daily for eight days, followed by a five day dosing holiday. In some embodiments, dosages are administered daily for eight days, followed by a six day dosing holiday. In some embodiments, dosages are administered daily for eight days, followed by a seven day dosing holiday. In some embodiments, dosages are administered daily for eight days, followed by an eight day dosing holiday. In some embodiments, dosages are administered daily for eight days, followed by a nine day dosing holiday. In some embodiments, dosages are administered daily for eight days, followed by a ten day dosing holiday. In some embodiments, dosages are administered daily for eight days, followed by an eleven day dosing holiday. In some embodiments, dosages are administered daily for eight days, followed by a twelve day dosing holiday. In some embodiments, dosages are administered daily for eight days, followed by a thirteen day dosing holiday. In some embodiments, dosages are administered daily for eight days, followed by a fourteen day dosing holiday.

In some embodiments, dosages are administered daily for nine days, followed by a one day dosing holiday. In some embodiments, dosages are administered daily for nine days, followed by a two day dosing holiday. In some embodiments, dosages are administered daily for nine days, followed by a three day dosing holiday. In some embodiments, dosages are administered daily for nine days, followed by a four day dosing holiday. In some embodiments, dosages are administered daily for nine days, followed by a five day dosing holiday. In some embodiments, dosages are administered daily for nine days, followed by a six day dosing holiday. In some embodiments, dosages are administered daily for nine days, followed by a seven day dosing holiday. In some embodiments, dosages are administered daily for nine days, followed by an eight day dosing holiday. In some embodiments, dosages are administered daily for nine days, followed by a nine day dosing holiday. In some embodiments, dosages are administered daily for nine days, followed by a ten day dosing holiday. In some embodiments, dosages are administered daily for nine days, followed by an eleven day dosing holiday. In some embodiments, dosages are administered daily for nine days, followed by a twelve day dosing holiday. In some embodiments, dosages are administered daily for nine days, followed by a thirteen day dosing holiday. In some embodiments, dosages are administered daily for nine days, followed by a fourteen day dosing holiday.

In some embodiments, dosages are administered daily for ten days, followed by a one day dosing holiday. In some embodiments, dosages are administered daily for ten days, followed by a two day dosing holiday. In some embodiments, dosages are administered daily for ten days, followed by a three day dosing holiday. In some embodiments, dosages are administered daily for ten days, followed by a four day dosing holiday. In some embodiments, dosages are administered daily for ten days, followed by a five day dosing holiday. In some embodiments, dosages are administered daily for ten days, followed by a six day dosing holiday. In some embodiments, dosages are administered daily for ten days, followed by a seven day dosing holiday. In some embodiments, dosages are administered daily for ten days, followed by an eight day dosing holiday. In some embodiments, dosages are administered daily for ten days, followed by a nine day dosing holiday. In some embodiments, dosages are administered daily for ten days, followed by a ten day dosing holiday. In some embodiments, dosages are administered daily for ten days, followed by an eleven day dosing holiday. In some embodiments, dosages are administered daily for ten days, followed by a twelve day dosing holiday. In some embodiments, dosages are administered daily for ten days, followed by a thirteen day dosing holiday. In some embodiments, dosages are administered daily for ten days, followed by a fourteen day dosing holiday.

In some embodiments, dosages are administered daily for eleven days, followed by a one day dosing holiday. In some embodiments, dosages are administered daily for eleven days, followed by a two day dosing holiday. In some embodiments, dosages are administered daily for eleven days, followed by a three day dosing holiday. In some embodiments, dosages are administered daily for eleven days, followed by a four day dosing holiday. In some embodiments, dosages are administered daily for eleven days, followed by a five day dosing holiday. In some embodiments, dosages are administered daily for eleven days, followed by a six day dosing holiday. In some embodiments, dosages are administered daily for eleven days, followed by a seven day dosing holiday. In some embodiments, dosages are administered daily for eleven days, followed by an eight day dosing holiday. In some embodiments, dosages are administered daily for eleven days, followed by a nine day dosing holiday. In some embodiments, dosages are administered daily for eleven days, followed by a ten day dosing holiday. In some embodiments, dosages are administered daily for eleven days, followed by an eleven day dosing holiday. In some embodiments, dosages are administered daily for eleven days, followed by a twelve day dosing holiday. In some embodiments, dosages are administered daily for eleven days, followed by a thirteen day dosing holiday. In some embodiments, dosages are administered daily for eleven days, followed by a fourteen day dosing holiday.

In some embodiments, dosages are administered daily for twelve days, followed by a one day dosing holiday. In some embodiments, dosages are administered daily for twelve days, followed by a two day dosing holiday. In some embodiments, dosages are administered daily for twelve days, followed by a three day dosing holiday. In some embodiments, dosages are administered daily for twelve days, followed by a four day dosing holiday. In some embodiments, dosages are administered daily for twelve days, followed by a five day dosing holiday. In some embodiments, dosages are administered daily for twelve days, followed by a six day dosing holiday. In some embodiments, dosages are administered daily for twelve days, followed by a seven day dosing holiday. In some embodiments, dosages are administered daily for twelve days, followed by an eight day dosing holiday. In some embodiments, dosages are administered daily for twelve days, followed by a nine day dosing holiday. In some embodiments, dosages are administered daily for twelve days, followed by a ten day dosing holiday. In some embodiments, dosages are administered daily for twelve days, followed by an eleven day dosing holiday. In some embodiments, dosages are administered daily for twelve days, followed by a twelve day dosing holiday. In some embodiments, dosages are administered daily for twelve days, followed by a thirteen day dosing holiday. In some embodiments, dosages are administered daily for twelve days, followed by a fourteen day dosing holiday.

In some embodiments, dosages are administered daily for thirteen days, followed by a one day dosing holiday. In some embodiments, dosages are administered daily for thirteen days, followed by a two day dosing holiday. In some embodiments, dosages are administered daily for thirteen days, followed by a three day dosing holiday. In some embodiments, dosages are administered daily for thirteen days, followed by a four day dosing holiday. In some embodiments, dosages are administered daily for thirteen days, followed by a five day dosing holiday. In some embodiments, dosages are administered daily for thirteen days, followed by a six day dosing holiday. In some embodiments, dosages are administered daily for thirteen days, followed by a seven day dosing holiday. In some embodiments, dosages are administered daily for thirteen days, followed by an eight day dosing holiday. In some embodiments, dosages are administered daily for thirteen days, followed by a nine day dosing holiday. In some embodiments, dosages are administered daily for thirteen days, followed by a ten day dosing holiday. In some embodiments, dosages are administered daily for thirteen days, followed by an eleven day dosing holiday. In some embodiments, dosages are administered daily for thirteen days, followed by a twelve day dosing holiday. In some embodiments, dosages are administered daily for thirteen days, followed by a thirteen day dosing holiday. In some embodiments, dosages are administered daily for thirteen days, followed by a fourteen day dosing holiday.

In some embodiments, dosages are administered daily for fourteen days, followed by a one day dosing holiday. In some embodiments, dosages are administered daily for fourteen days, followed by a two day dosing holiday. In some embodiments, dosages are administered daily for fourteen days, followed by a three day dosing holiday. In some embodiments, dosages are administered daily for fourteen days, followed by a four day dosing holiday. In some embodiments, dosages are administered daily for fourteen days, followed by a five day dosing holiday. In some embodiments, dosages are administered daily for fourteen days, followed by a six day dosing holiday. In some embodiments, dosages are administered daily for fourteen days, followed by a seven day dosing holiday. In some embodiments, dosages are administered daily for fourteen days, followed by an eight day dosing holiday. In some embodiments, dosages are administered daily for fourteen days, followed by a nine day dosing holiday. In some embodiments, dosages are administered daily for fourteen days, followed by a ten day dosing holiday. In some embodiments, dosages are administered daily for fourteen days, followed by an eleven day dosing holiday. In some embodiments, dosages are administered daily for fourteen days, followed by a twelve day dosing holiday. In some embodiments, dosages are administered daily for fourteen days, followed by a thirteen day dosing holiday. In some embodiments, dosages are administered daily for fourteen days, followed by a fourteen day dosing holiday.

In some embodiments, dosages are administered daily for thirty days followed by a thirty day dosing holiday. In some embodiments, dosages are administered daily for thirty days followed by a 25-30 day dosing holiday. In some embodiments, dosages are administered daily for thirty days followed by a 20-25 day dosing holiday. In some embodiments, dosages are administered daily for thirty days followed by a 15-20 day dosing holiday. In some embodiments, dosages are administered daily for thirty days followed by a 10-15 day dosing holiday. In some embodiments, dosages are administered daily for thirty days followed by a 5-10 day dosing holiday. In some embodiments, dosages are administered daily for thirty days followed by a 1-5 day dosing holiday.

In some embodiments, dosages are administered daily for 25-30 days followed by a thirty day dosing holiday. In some embodiments, dosages are administered daily for 25-30 days followed by a 25-30 day dosing holiday. In some embodiments, dosages are administered daily for 25-30 days followed by a 20-25 day dosing holiday. In some embodiments, dosages are administered daily for 25-30 days followed by a 15-20 day dosing holiday. In some embodiments, dosages are administered daily for 25-30 days followed by a 10-15 dosing holiday. In some embodiments, dosages are administered daily for 25-30 days followed by a 5-10 day dosing holiday. In some embodiments, dosages are administered daily for 25-30 days followed by a 1-5 day dosing holiday.

In some embodiments, dosages are administered daily for 20-25 days followed by a thirty day dosing holiday. In some embodiments, dosages are administered daily for 20-25 days followed by a 25-30 day dosing holiday. In some embodiments, dosages are administered daily for 20-25 days followed by a 20-25 day dosing holiday. In some embodiments, dosages are administered daily for 20-25 days followed by a 15-20 day dosing holiday. In some embodiments, dosages are administered daily for 20-25 days followed by a 10-15 dosing holiday. In some embodiments, dosages are administered daily for 20-25 days followed by a 5-10 day dosing holiday. In some embodiments, dosages are administered daily for 20-25 days followed by a 1-5 day dosing holiday.

In some embodiments, dosages are administered daily for 15-20 days followed by a thirty day dosing holiday. In some embodiments, dosages are administered daily for 15-20 days followed by a 25-30 day dosing holiday. In some embodiments, dosages are administered daily for 15-20 days followed by a 20-25 day dosing holiday. In some embodiments, dosages are administered daily for 15-20 days followed by a 15-20 day dosing holiday. In some embodiments, dosages are administered daily for 15-20 days followed by a 10-15 day dosing holiday. In some embodiments, dosages are administered daily for 15-20 days followed by a 5-10 day dosing holiday. In some embodiments, dosages are administered daily for 15-20 days followed by a 1-5 day dosing holiday.

in any of the foregoing embodiments, the daily dosing may be administered in one dose administered once or day, or in two or more divided doses administered multiple times per day. For example, the compounds described herein may be administered once per day, twice per day, three times per day, or four times per day.

In some embodiments, the subject's thyroid hormone levels, T3, T4 or TSH levels are monitored, such that administration of a daily dose can be eliminated or reduced on any day in which the T3, T4, or TSH levels are below a pre-determined threshold. When the T3, T4, or TSH levels rise above a pre-determined threshold during the dosing holiday, normal daily dosing can continue.

### Dosages of Administration

A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

In one aspect, the compound of formula I and the at least one additional therapeutic agent (e.g., additional therapeutic agents described herein) are administered at dosages substantially the same as the dosages at which they are administered in the respective monotherapies. In one aspect, the compound of formula I is administered at a dosage which is less than (e.g., less than 90%, less than 80%), less than 70%, less than 60%, less than 50, less than 40%, less than 30%, less than 20%, or less than 10% its monotherapy dosage. In one aspect, the at least one additional therapeutic agent (e.g., additional therapeutic agents described herein) is administered at a dosage which is less than (e.g., less than 90%, less than 80%, less than 70%, less than 60%, less than 50, less than 40%, less than 30%, less than 20%, or less than 10%) its monotherapy dosage. In one aspect, both the first compound and the at least one additional therapeutic agent (e.g., additional therapeutic agents described herein) are administered at a dosage which is less than (e.g., less than 90%, less than 80%, less than 70%, less than 60%, less than 50, less than 40%, less than 30%, less than 20%, or less than 10%) their respective monotherapy dosages.

The actual unit dose of the active compounds described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight.

In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, from about 20 mg to about 300 mg, or from about 200 mg to about 2000 mg.

In some embodiments, the actual unit dose is 5 mg. In some embodiments the actual unit dose is 10 mg. In some embodiments, the actual unit dose is 25 mg. In some embodiments, the actual unit dose is 250 mg or less. In some embodiments, the actual unit dose is 100 ng or less. In some embodiments, the actual unit dose is 70 mg or less. In some embodiments, the actual unit does is 5 mg.

In some embodiments, the mode of administration comprises administering a loading dose followed by a maintenance dose. In some embodiments, the loading dose is 300 mg or less; 250 mg or less, 200 mg or less, 150 mg or less, or 100 mg or less. In some embodiments, the maintenance dose is 300 mg or less; 200 mg or less, 100 mg or less, 50 mg or less, 40 mg or less, 25 mg or less, 10 mg or less, 5 mg or less, or 1 mg or less.

In some embodiments the loading dose is administered over a period of one day. In some embodiments the loading dose is administered over a period of 2 days. In some embodiments the loading dose is administered over a period of 3 days. In some embodiments the loading dose is administered over a period of 4 days. In some embodiments the loading dose is administered over a period of 5, 6 or 7 days. In some embodiments, the loading dose is administered over a period of 8-14 days or fewer. In some embodiments, the loading dose is administered over a period of 14 days.

Administration of the active agents described herein may be achieved by modulating the dosing schedule such that subjects experience periodic partial or full reductions in dosing for fixed amounts of time, followed by a resumption of dosing.

In some embodiments, dosages are administered daily for between one and thirty days, followed by a dosing holiday lasting for between one and thirty days.

In some embodiments, during the dosing holiday, no dose is administered.

In further embodiments, the compound of formula I and its metabolites are allowed to clear completely from the subject's body prior to administration of the next dose.

In some other embodiments, during the dosing holiday, a dose less than the usual daily dose is administered.

In some embodiments, the maximum serum concentration of the compound of formula 1 during the dosing schedule is less than 120 ng/ml, less than 100 ng/ml, less than 90 ng/ml, less than 80 ng/ml, less than 70 ng/ml, less than 60 ng/ml, or less than 50 ng/ml.

In some embodiments, the minimum serum concentration during the dosing schedule is less than 10 ng/ml, less than 1 ng/ml, less than 0.1 ng/ml, less than 0.01 ng/ml, or less than 0.001 ng/ml.

In some embodiments, the maximum serum concentration of the compound of formula I during the initial (loading) phase of administration is less than 500 ng/ml, less than 400 ng/ml, less than 300 ng/ml, less than 200 ng/ml, less than 150 ng/ml, less than 120 ng/ml, less than 100 ng/ml, less than 90 ng/ml, less than 80 ng/ml, less than 70 ng/ml, less than 60 ng/ml, or less than 50 ng/ml.

In some such embodiments, the maximum serum concentration during the initial phase of administration is from 5 ng/ml to 250 ng/ml. In some embodiments, the maximum serum concentration of the compound of formula I during the subsequent (maintenance) phase of administration is less than 350 ng/ml, less than 200 ng/ml, less than 120 ng/ml, less than 100 ng/ml, less than 90 ng/ml, less than 80 ng/ml, less than 70 ng/ml, less than 60 ng/ml, or less than 50 ng/ml, less than 40 ng/ml, less than 35 ng/ml, or less than 10 ng/ml.

One of ordinary skill in the art will readily be aware of such methods as exist in the art for the monitoring of serum concentrations of pharmaceutical agents, and means of adjusting dosages of the compounds disclosed herein in order to achieve the desired serum concentrations. In some embodiments, the weekly dose to be administered is 600 mg or less.

In some embodiments, the weekly dose is to be administered is 500 mg or less, 400 mg or less, 300 mg or less, 200 mg or less, 100 mg or less, 50 mg or less, 40 mg or less, 25 mg or less, 10 mg or less, or 5 mg or less, or within a range defined by any two of the foregoing.

According to the present disclosure, the dosing schedule may be varied in order to attain the desired therapeutic effect. In particular, variations in the dosing schedule as described may be repeated throughout the duration of the treatment.

For example, in certain embodiments, the first dosage may be higher, lower, or the same as the dosages following the first dosage. In addition, a loading dose may precede the disclosed dosing regimen, and a dosing holiday may or may not follow the administration of the loading dose.

The present application is further illustrated by the following examples that should not be construed as limiting. The contents of all references, patents, and published patent applications cited throughout this application, as well as the Figures and Tables, are incorporated herein by reference.

### EXAMPLES

### Example 1

### Composition of Formulation

TABLE 1
Prescription composition of Example
1 Prescription Formulation number

| | Formulation No. | | |
|---|---|---|---|
| Composition prescription (mg) | A1 | B1 | C1 |
| Compound represented by formula (I) | 10 | 2 | 5 |
| Polyvinyl caprolactam-polyvinyl acetate- | 220 | 66 | 165 |
| polyethylene glycol graft copolymer | | | |
| (Soluplus ®) | | | |
| triethyl citrate | 0 | 0 | 5 |
| Weight of extrudate | 230 | 68 | 175 |
| Additional accessories | A1 | B1 | C1 |
| mannitol | 80 | 35 | 55 |
| Cross-linked sodium carboxymethyl cellulose | 25 | 10 | 10 |
| Colloidal silica | 2 | 1 | 1 |
| magnesium stearate | 3 | 1 | 2 |
| Total additional excipients | 110 | 47 | 68 |
| Total Amounts | 340 | 115 | 243 |

### Preparation Process:

1. Pretreatment of API and excipients: the API and excipients to be used in prescription research are crushed, sieved and dried by the conventional means of preparation technology to remove caking during storage and reduce the moisture content of hygroscopic excipients so as to meet the standards for further preparation;

2. Ingredients: API and excipients for hot melt extrusion are weighed according to prescription proportion and preparation scale;

3. Mixing: mixing API and excipients with finished ingredients uniformly by conventional means of preparation technology;

4. Hot melt extrusion: setting the extrusion temperature for different areas of the extruder; After preheating to the set temperature, keep the temperature for 15 min-30 min, uniformly add the uniformly mixed API and excipients in the way of manual feeding or using loss in weight loss feeder, and extrude at the preset extrusion speed; By adjusting the temperature of different areas of the extruder barrel, screw rotation speed and feeding speed, the extrusion die temperature is controlled between 90° C. and 130° C., the screw torque is kept in a stable range, and the extruded material is transparent; Adjust the extrusion speed and feeding speed to control the residence time of materials in the barrel of hot melt extruder within 30 min;

5. Crushing the extrudate: Crushing the cooled extrudate by conventional means of preparation technology;

6. Total mixing: according to the prescription proportion, add other excipients, and mix the above materials by the conventional mixing means of preparation technology;

7. Preparation: Formulas A1 and C1 are pressed into capsule-shaped tablets. The total mixture of prescription B1 was filled into Vcaps Plus® (Size 3) hydroxypropyl methylcellulose capsule;

8. Packaging: put the tablets of prescription A1 and B1 and the capsules of prescription B1 into HDPE bottles, and seal them with aluminum film;

9. Preservation: the packaged tablets or capsules of the compound shown in formula (I) are stored at room temperature (Less than 30° C.).

### Comparative Example 1

### Composition of Formulation

TABLE 2
Formulation composition of Comparative Example 2

| | Formulation No. |
|---|---|
| Composition prescription (mg) | a2 |
| Compound represented by formula (I) | 5 |
| Covidone Kollidon VA64 | 165 |
| Polyethylene glycol 6000 | 5 |
| extrudate | 175 |
| Additional accessories | a2 |
| mannitol | 55 |
| Cross-linked sodium carboxymethyl | 10 |
| cellulose | |
| Colloidal silica | 1 |
| magnesium stearate | 2 |
| Total additional excipients | 68 |
| Total Amounts | 243 |

### Preparation Process:

1. Pretreatment of API and excipients: the API and excipients to be used in prescription research are crushed, sieved and dried by the conventional means of preparation technology to remove caking during storage and reduce the moisture content of hygroscopic excipients so as to meet the standards for further preparation;

2. Ingredients: API and excipients for hot melt extrusion are weighed according to prescription proportion and preparation scale;

3. Mixing: mixing API and excipients with finished ingredients evenly by the conventional means of preparation technology;

4. Hot melt extrusion: setting the extrusion temperature for different areas of the extruder; After preheating to the set temperature, keep the temperature for 15 min-30 min, uniformly add the uniformly mixed API and excipients in the way of manual feeding or using loss in weight feeder, and extrude at the preset extrusion speed; By adjusting the temperature of different areas of the extruder barrel, screw rotation speed and feeding speed, the temperature of the extrusion die is controlled between 100° C. and 130° C., the screw torque is kept in a stable range, and the extruded material is transparent; Adjust the extrusion speed and feeding speed to control the residence time of materials in the barrel of hot melt extruder within 30 min;

5. Crushing the extrudate: Crushing the cooled extrudate by conventional means of preparation technology;

6. Total mixing: according to the prescription proportion, add other excipients, and mix the above materials by the conventional mixing means of preparation technology;

7. Preparation: formula b2 is compressed into capsule-shaped tablets;

8. Packaging: put the tablet of formula b2 into a HDPE bottle and seal it with aluminum film;

9. Preservation: the packaged tablets or capsules of the compound shown in formula (I) are stored at room temperature (Less than 30° C.).

### Effect Example 1

In this example, the reasons for choosing the proportion and preparation process of the composition of the present invention are illustrated by the dissolution test design with increasing pH for 24 hours.

Take the granules obtained by hot melt extrusion according to formula A1-C1 of Example 1 and the granules obtained by hot melt extrusion according to formula a2 of Comparative Example 1, and investigate the pH transition and supersaturation maintenance time of simulated human digestive juice.

The dissolution conditions were as follows: firstly, 750 mL degassed hydrochloric acid solution with pH 2.0 at 37° C.±0.5° C. was used as dissolution medium, and then dissolved with stirring at 50 rpm by paddle method for 2 h, then 250 mL degassed phosphate buffer solution with pH6.8 concentration was added, and then dissolved with stirring at 50 rpm by paddle method for 22 h (USP Apparatus II). The granules were directly and accurately weighed and then put in, and samples were taken at 30, 60, 90, 120, 180, 240, 360, 480, 720, 960, 1200 and 1440 min after putting in, and the subsequent filtrate was taken and diluted with 75% acetonitrile water solution in equal proportion, and the concentration of the compound shown in formula (I) was determined by HPLC

Determination conditions of HPLC: Octadecylsilane bonded silica gel was selected as the packing (Welch Ultimate® XB-C18 4.6*150 mm, 5 μm, or equivalent column), 0.05% trifluoroacetic acid aqueous solution-acetonitrile (30:70) was used as the mobile phase, the flow rate was 1.0 ml/min, the column temperature was 30° C., and the detection wavelength was 230 nm®. Accurately measure the reference solution and the test solution (in which the injection volume of 2 mg prescription is 40 μl, and the injection volume of 5 mg and 10 mg prescription is 20 μl), and inject them into the liquid chromatograph respectively, record the chromatogram, and calculate the dissolution according to the peak area according to the external standard method.

### Results:

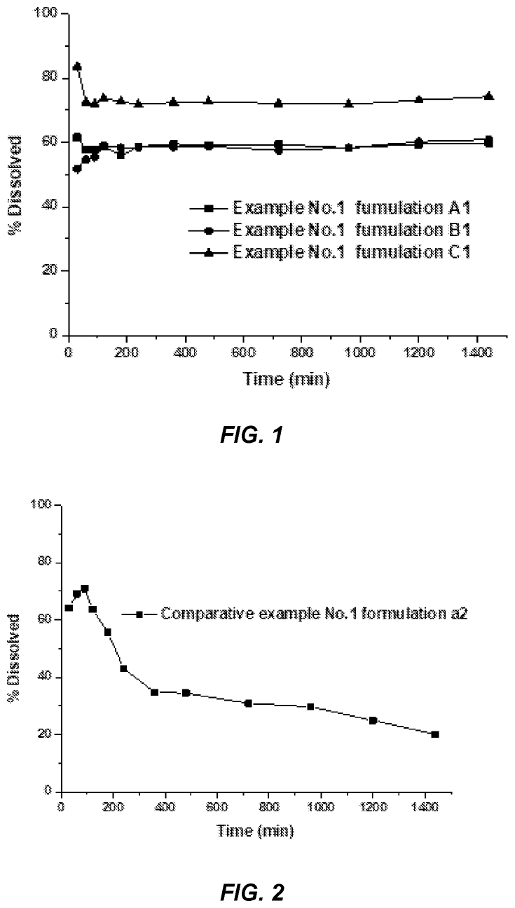

I. As shown in Table 3 and FIG. 1, the compound shown in formula (I) of the extrudate prepared according to the proportion of Example 1 of the present invention can realize stable supersaturated dissolution within 24 h, and the dissolution at the end of 24 h is 88.8%˜100.0% relative to the dissolution at the highest point.

11. As shown in table 4 and FIG. 2, the compound shown in formula (I) in comparative example 1 had obvious supersaturation precipitation during dissolution, and the dissolution rate at the end of 24 h was only 28.3% compared with the dissolution rate at the highest point.

III. Calculation of the area under the dissolution curve, which is 171.2%(83104.5/48549.0*100%), 170.2%(82648.5/48549.0*100%) and 211.0% (102456.0/48556.0%) of that of the comparative example 1, respectively

### Conclusion

The dissolution comparison results of Example 1 and Comparative Example 1 show that the technical scheme described in Example 1 has special advantages in maintaining the stable supersaturated concentration of the compound shown in formula (I) for a long time.

TABLE 3
Dissolution results of different prescription
samples in Example 1 (n = 6)

| | |
|---|---|
| Time | Dissolution (Mean ± SD, %) |

| min | A1 | B1 | C1 |
|---|---|---|---|
| 30 | 61.6 ± 13.2 | 51.7 ± 3.9 | 83.6 ± 12.5 |
| 60 | 57.8 ± 1.5 | 54.7 ± 3.4 | 72.5 ± 1.8 |
| 90 | 57.6 ± 1.8 | 55.4 ± 2.0 | 72.0 ± 1.5 |
| 120 | 58.7 ± 0.6 | 59.0 ± 1.8 | 73.6 ± 2.6 |
| 180 | 55.9 ± 0.7 | 58.3 ± 2.0 | 72.7 ± 2.3 |
| 240 | 58.7 ± 0.8 | 58.5 ± 2.7 | 71.7 ± 2.3 |
| 360 | 59.4 ± 0.5 | 58.5 ± 3.2 | 72.4 ± 2.2 |
| 480 | 59.2 ± 0.4 | 58.8 ± 1.5 | 72.6 ± 2.1 |
| 720 | 59.3 ± 0.8 | 57.5 ± 3.3 | 72.1 ± 2.2 |
| 960 | 58.3 ± 0.6 | 58.3 ± 0.9 | 71.8 ± 2.1 |
| 1200 | 59.4 ± 0.7 | 60.2 ± 2.6 | 73.1 ± 2.3 |
| 1440 | 59.8 ± 0.6 | 60.9 ± 2.2 | 74.2 ± 2.6 |
| Area under curve | 83104.5 | 82648.5 | 102456.0 |
| Dissolution at | 97.1% | 100.0% | 88.8% |
| 1440 min/maximum | | | |
| dissolution (%) | | | |

TABLE 4
Dissolution results of different prescription
samples in Example 2 (n = 6)

| Time | Dissolution (Mean ± SD, %) |
|---|---|
| (min) | a2 |
| 30 | 64.1 ± 3.0 |
| 60 | 69.1 ± 4.2 |
| 90 | 70.9 ± 5.6 |
| 120 | 63.7 ± 7.2 |
| 180 | 55.6 ± 9.6 |
| 240 | 43.1 ± 15.5 |
| 360 | 34.7 ± 14.2 |
| 480 | 34.5 ± 13.1 |
| 720 | 30.9 ± 13.9 |
| 960 | 29.7 ± 3.3 |
| 1200 | 24.9 ± 6.0 |
| 1440 | 20.1 ± 4.2 |
| Area under curve | 48549.0 |
| Dissolution at | 28.3% |
| 1440 min/maximum | |
| dissolution (%) | |

### Effect Example 2

Because the concentration of micelle system made of the compound shown in (I) and carrier excipients in 900 mL dissolution cup is too dilute, it is difficult to determine by laser particle size method. The micelle sizes that may be formed in Example 1 and Comparative Example 1 were compared by the following improved method.

The extrudate powder of example 1 and comparative example 1 containing 5 mg of the compound shown in formula (I) is put into a 100 mL volumetric flask, and then added with pH6.0 phosphate buffer solution with a concentration of 50 mM at 37.0° C.±0.5° C. for constant volume, and shaken in a water bath at 37.0° C.*0.5° C. for 15 min to fully dissolve and disperse. Samples were determined by NICOMP™ 380 ZLS laser particle size Analyzer™. The particle size distribution of micelles was calculated with the Intensity of Gaussian Distribution, and the results are shown in Table 5.

The results of particle size measurement show that the nano-micelle prepared by the scheme of Example 1 has smaller particle size and more concentrated distribution compared with the micelle prepared by the scheme of Comparative Example 1, suggesting that it has better stability.

TABLE S
Particle size measurement results

| | | | | Comparative |
|---|---|---|---|---|
| | Example 1 | Example 1 | Example 1 | example 1 |
| | Form. | Form. | Form. | Form. |
| Particle size measurement results | A1 sample | B1 sample | C1 sample | A2 sample |
| Average particle size (nm, nanometer) | 63.75 | 57.77 | 57.79 | 126.43 |
| Standard deviation (St. Dev) | 20.15 | 9.36 | 14.51 | 48.30 |
| Coefficient of variation (CV %) | 31.60% | 16.20% | 25.10% | 38.20% |
| Polydispersion coefficient (PI) | 0.10 | 0.03 | 0.06 | 0.15 |

1. A pharmaceutical composition, comprising by weight:
(1) 1 part of a compound of formula (I) 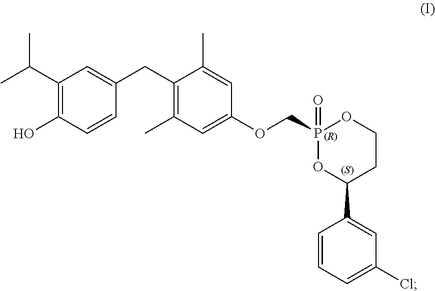
(2) 9 to 45 parts of polymer material,
wherein the compound of formula (I) maintains a supersaturated concentration in the pharmaceutical composition and is suitable for storage at room temperature.

2. The pharmaceutical composition of claim 1, wherein the polymer material comprises one or more of the polymers selected from the group consisting of polyvinyl lactam polymers, cellulose derivative polymers, polyacrylic resins, polyethylene glycol, polyoxyethylene and polyvinyl alcohol.

3. The pharmaceutical composition of claim 1, wherein the polymer material comprises one or more polyvinyl lactam polymers selected from the group consisting of polyvinylpyrrolidone (PVP), polyvinylpyrrolidone/vinyl acetate (PVP/VA) and polyethylene caprolactam-polyvinyl acetate-polyethylene glycol graft copolymers.

4. The pharmaceutical composition of claim 1, wherein the polymer material comprises polyvinyl caprolactam-polyvinyl acetate-polyethylene glycol graft copolymers

5. The pharmaceutical composition of claim 1, wherein the pharmaceutical composition comprises nano micelles.

6. The pharmaceutical composition of claim 1, wherein the pharmaceutical composition is in the form of an amorphous solid dispersion prepared by hot melt extrusion.

7. The pharmaceutical composition of claim 1, wherein the composition is formulated in a fixed dose tablet or capsule that contains 1-15 mg of the compound of formula (I).

8. The pharmaceutical composition of claim 7, wherein the fixed dose tablet or capsule contains 2.5 mg or 5 mg of the compound of formula (I).

9. The pharmaceutical composition of claim 1, further comprising one or more non-polymeric material selected from the group consisting of pharmaceutically acceptable pharmaceutical excipients, nonvolatile weak acids, neutral or weakly acidic inorganic substances, and pharmaceutically acceptable excipients with a melting point lower than 80° C.

10. The pharmaceutical composition of claim 1, wherein the compound of formula (I) is present in the pharmaceutical composition in a crystalline form, an amorphous form, or a part-crystalline/part amorphous form.

11. The pharmaceutical composition of claim 10, wherein the amorphous form of the compound of formula (I) is present in the pharmaceutical composition in an amount of 50% or greater by weight of the total amount of the compound of formula (I).

12. The pharmaceutical composition of claim 10, wherein the amorphous form of the compound of formula (I) is present in the pharmaceutical composition in an amount of 80% or greater by weight of the total amount of the compound of formula (I).

13. The pharmaceutical composition of claim 10, wherein the amorphous form of the compound of formula (I) is present in the pharmaceutical composition in an amount of 90% or greater by weight of the total amount of the compound of formula (I).

14. The pharmaceutical composition of claim 1, wherein the composition is formulated in an extended-release or delayed-release formulation.

15. A method for treatment of disease in a patient in need thereof, comprising:
administering to the patient an effective amount of the pharmaceutical composition of claim 1.

16. The method of claim 15, wherein the disease is selected from the group consisting of nonalcoholic fatty liver disease (NAFLD) and non-alcoholic steatohepatitis (NASH).

17. A method of preparing a pharmaceutical composition, comprising the steps of:
extruding a pre-extrusion mixture comprising by weight (1) 1 part of a compound of formula (I) and (2) 9 to 45 parts of polymer material by hot melt extrusion to form an extruded product; and
cutting or grinding the extruded product into granules or powders,
wherein the compound of formula (I) maintains a supersaturated concentration in the extruded product at room temperature.

18. The method of claim 17, further comprising the step of:
compressing the granules or powders into fixed dose tablets.

19. The method of claim 17, wherein the extruding step is performed at an extrusion temperature of 90-130° C.

20. The method of claim 1, wherein the polymer material comprises polyethylene caprolactam-polyvinyl acetate-polyethylene glycol graft copolymers.
